(19) United States
(12) Patent Application Publication
(10) Pub. No.: US 2016/0106939 A1
(43) Pub. Date: Apr. 21, 2016

(54) EXPANDABLE INTUBATION ASSEMBLIES

(71) Applicant: Talal Sharaiha LLC, Las Vegas, NV (US)

(72) Inventors: Talal Sharaiha, New York, NY (US); Nikhil Ramchandra Katre, Maharashtra (IN); Rajesh Tulsiram Shelke, Maharashtra (IN); Sandeep Jagannath Walde, Maharashtra (IN)

(21) Appl. No.: 14/622,123

(22) Filed: Feb. 13, 2015

Related U.S. Application Data
(60) Provisional application No. 62/066,145, filed on Oct. 20, 2014.

(51) Int. Cl. A61M 16/04

(52) U.S. Cl. CPC A61M 16/0465; A61M 16/0488

(57) ABSTRACT

Expandable intubation assemblies and methods for using and making the same are provided. In one example embodiment, an intubation assembly includes a first tube, a second tube, and an expander coupled to the first tube, where movement of the second tube with respect to the first tube along the length of the assembly adjusts a cross-sectional dimension of the expander. Additional embodiments are also provided.

### CROSS-REFERENCE TO RELATED APPLICATION(S)

This application claims the benefit of prior filed U.S. Provisional Patent Application No. 62/066,145, filed Oct. 20, 2014, which is hereby incorporated by reference herein in its entirety.

### TECHNICAL FIELD

This disclosure relates to expandable assemblies and, more particularly, to expandable intubation assemblies and methods for using and making the same.

### BACKGROUND OF THE DISCLOSURE

Various medical procedures (e.g., intubation procedures) involve a distal end of a tube being inserted into a specific area of a patient and then using the tube for injecting material into the patient and/or for removing material from the patient. However, safely securing such a tube at a particular position within the patient during use has heretofore been infeasible. Moreover, safely preventing certain material from passing along the external surface of such a tube during use has heretofore been infeasible.

### SUMMARY OF THE DISCLOSURE

This document describes expandable assemblies and methods for using and making the same.

As an example, an intubation assembly may include a first tube including a first tube passageway extending from a proximal first tube end to a distal first tube end along a first tube portion of the length of the assembly, and an expander including an expander passageway extending from a proximal expander end to a distal expander end along an expander portion of the length of the assembly. The proximal expander end is coupled to the distal first tube end and movement of the distal expander end with respect to the proximal expander end along the length of the assembly adjusts a cross-sectional dimension of the assembly.

As another example, an intubation assembly may include a first tube including a first tube passageway extending from a proximal first tube end to a distal first tube end along a first tube portion of the length of the assembly, a second tube including a second tube passageway extending from a proximal second tube end to a distal second tube end along a second tube portion of the length of the assembly, and an expander including an expander passageway extending from a proximal expander end to a distal expander end along an expander portion of the length of the assembly. The expander is coupled to the first tube, and movement of the second tube with respect to the first tube along the length of the assembly adjusts a cross-sectional dimension of the expander.

As yet another example, a method of intubating a patient with an assembly that includes a first tube, a second tube, and an expander coupled to the first tube, may include positioning the expander within the patient, after the positioning, moving the second tube with respect to the first tube for increasing a cross-sectional dimension of the expander, and, after the moving, passing fluid through the expander for treating the patient.

As yet another example, a method for intubating a target of a patient via a passageway of the patient with an assembly, where a length of the assembly extends between a proximal end and a distal end and includes an expander, may include inserting the distal end of the assembly in a first state of the assembly into the target, such that the expander is at least partially within one of the target and the passageway, and reconfiguring the inserted assembly from the first state of the assembly into a second state of the assembly, where the expander is in an unnatural state in the first state of the assembly, the expander is in a natural state in the second state of the assembly, and a cross-sectional dimension of the expander is larger in the second state of the assembly than in the first state of the assembly.

As yet another example, a method of intubating a patient with an assembly that includes an expander including an expander passageway extending from a proximal expander end to a distal expander end, may include positioning the expander within the patient, after the positioning, adjusting a distance between the proximal expander end and the distal expander end, and, after the adjusting, passing fluid through the expander passageway for treating the patient.

As yet another example, a method of intubating a patient with an assembly that includes an expander including an expander passageway extending from a proximal expander end to a distal expander end, may include applying a force to the assembly, wherein the applied force separates the distal expander end and the proximal expander end by an insertion dimension, during the applying, inserting the expander within the patient, and, after the inserting, terminating the applying, wherein the termination of the applied force enables the distal expander end to move towards the proximal expander end by an expansion dimension.

As yet another example, a method of intubating a patient with an intubation assembly including an inner tube, an outer tube, and an expander, may include positioning the expander about the inner tube at an expander position along the assembly, positioning the outer tube about the expander, inserting the expander position of the assembly within the patient, and moving the outer tube along the inner tube away from the expander position for reconfiguring the expander from a tensioned state to a relaxed state.

This Summary is provided merely to summarize some example embodiments, so as to provide a basic understanding of some aspects of the subject matter described in this document. Accordingly, it will be appreciated that the features described in this Summary are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The discussion below makes reference to the following drawings, in which like reference characters may refer to like parts throughout, and in which:

FIG. 1 is a cross-sectional view of a patient with an intubation assembly in an insertion state;

FIGS. 1A-1C are cross-sectional views, similar to FIG. 1, of the patient of FIG. 1 with the intubation assembly of FIG. 1 in various illustrative expanded states;

FIG. 1D is a cross-sectional view, similar to FIGS. 1-1C, of the patient of FIGS. 1-1C with the intubation assembly of FIGS. 1-1C in a removal state;

FIG. 2 is a side elevational view of the intubation assembly of FIGS. 1-1D in an expanded state;

FIG. 3 is a cross-sectional view of a portion of the intubation assembly of FIG. 2 in an insertion state;

FIG. 4 is a cross-sectional view of a portion of the intubation assembly of FIGS. 2 and 3 in an insertion state;

FIG. 4A is a perspective view of a portion of the intubation assembly of FIGS. 2-4 in an insertion state;

FIG. 5 is a cross-sectional view of a portion of the intubation assembly of FIGS. 2-4A in an expanded state;

FIG. 6 is a cross-sectional view of a portion of the intubation assembly of FIGS. 2-5 in an expanded state;

FIG. 6A is a perspective view of a portion of the intubation assembly of FIGS. 2-6 in an expanded state;

FIG. 6B is a side elevational view of a portion of the intubation assembly of FIGS. 2-6A, taken from line VIB-VIB of FIG. 6A;

FIG. 6C is a cross-sectional view of a portion of the intubation assembly of FIGS. 2-6B, taken from line VIC-VIC of FIG. 6B;

FIG. 6D is a perspective, but partially cut-away, view of a portion of the intubation assembly of FIGS. 2-6C in an expanded state;

FIG. 7 is a cross-sectional view, similar to FIG. 4, of a portion of another exemplary intubation assembly in an insertion state;

FIG. 8 is a cross-sectional view, similar to FIG. 5, of a portion of the intubation assembly of FIG. 7 in an expanded state;

FIG. 9 is a cross-sectional view, similar to FIGS. 4 and 7, of a portion of yet another exemplary intubation assembly in an insertion state;

FIG. 10 is a cross-sectional view, similar to FIGS. 5 and 8, of a portion of the intubation assembly of FIG. 9 in an expanded state;

FIG. 11 is a cross-sectional view, similar to FIGS. 4, 7, and 9, of a portion of yet another exemplary intubation assembly in an insertion state;

FIG. 12 is a cross-sectional view, similar to FIGS. 5, 8, and 10, of a portion of the intubation assembly of FIG. 11 in an expanded state;

FIG. 13A is a perspective view, similar to FIG. 6A, but of a portion of another exemplary intubation assembly in an expanded state;

FIG. 13B is a side elevational view of a portion of the intubation assembly of FIG. 13A, taken from line XIIIB-XIIIB of FIG. 13A;

FIG. 13C is a cross-sectional view of a portion of the intubation assembly of FIGS. 13A and 13B, taken from line XIIIC-XIIIC of FIG. 13B;

FIG. 14 is a side elevational view of another exemplary intubation assembly of FIGS. 1-1D in an expanded state;

FIG. 15 is a cross-sectional view of a portion of the intubation assembly of FIG. 14 in an insertion state;

FIG. 15A is a cross-sectional view of a portion of the intubation assembly of FIGS. 14 and 15 in an insertion state;

FIG. 15B is a cross-sectional view of a portion of the intubation assembly of FIGS. 14-15A in an insertion state;

FIG. 15C is a perspective view of a portion of the intubation assembly of FIGS. 14-15B in an insertion state;

FIG. 16 is a cross-sectional view of a portion of the intubation assembly of FIGS. 14-15C in an expanded state;

FIG. 16A is a perspective view of a portion of the intubation assembly of FIGS. 14-16 in an expanded state;

FIG. 17 is a cross-sectional view of a portion of the intubation assembly of FIGS. 14-16A in a removal state; and

FIGS. 18-22 are flowcharts of illustrative processes for intubating a patient.

### DETAILED DESCRIPTION OF THE DISCLOSURE

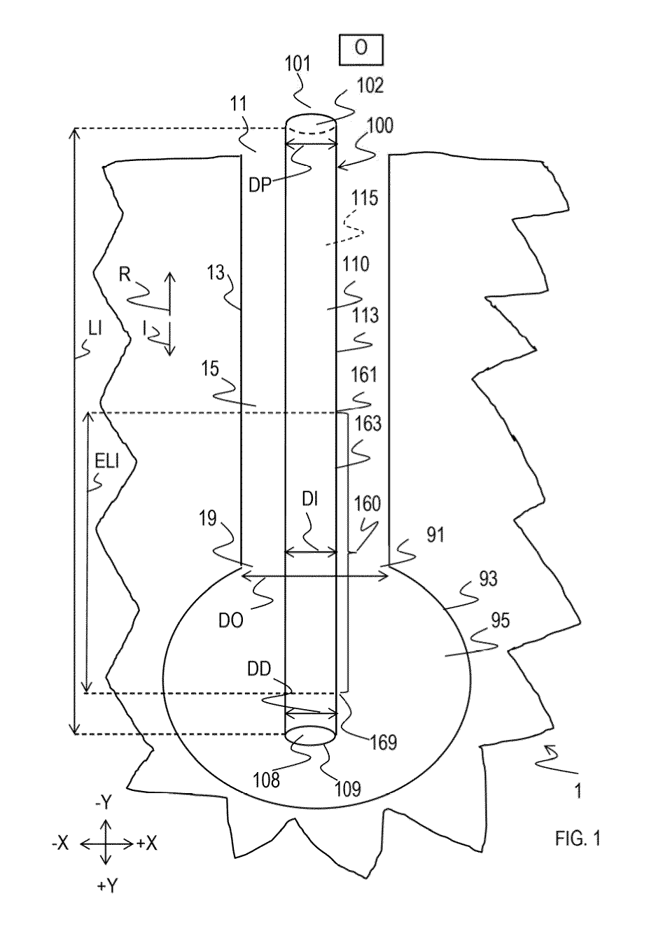

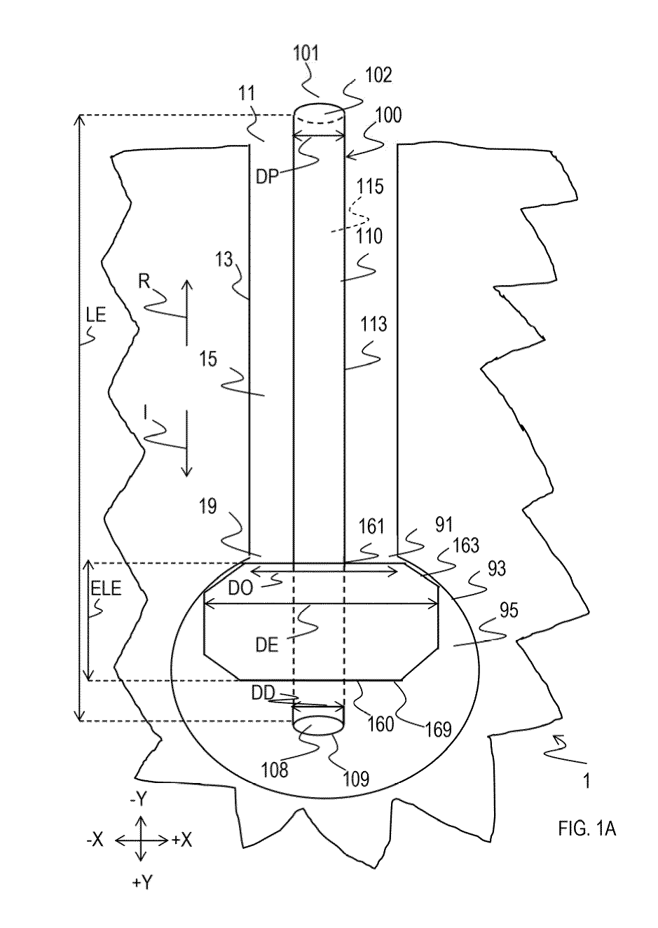

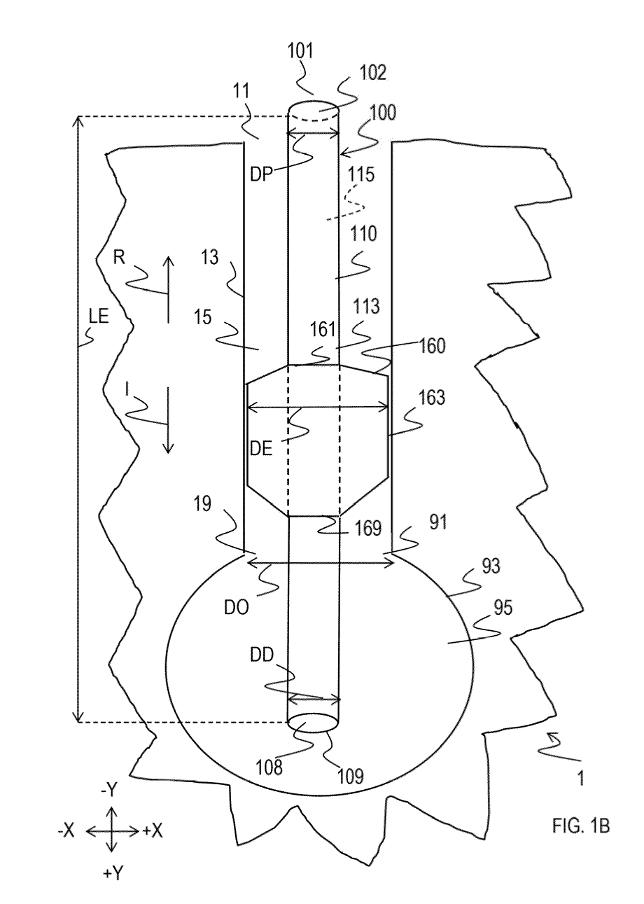

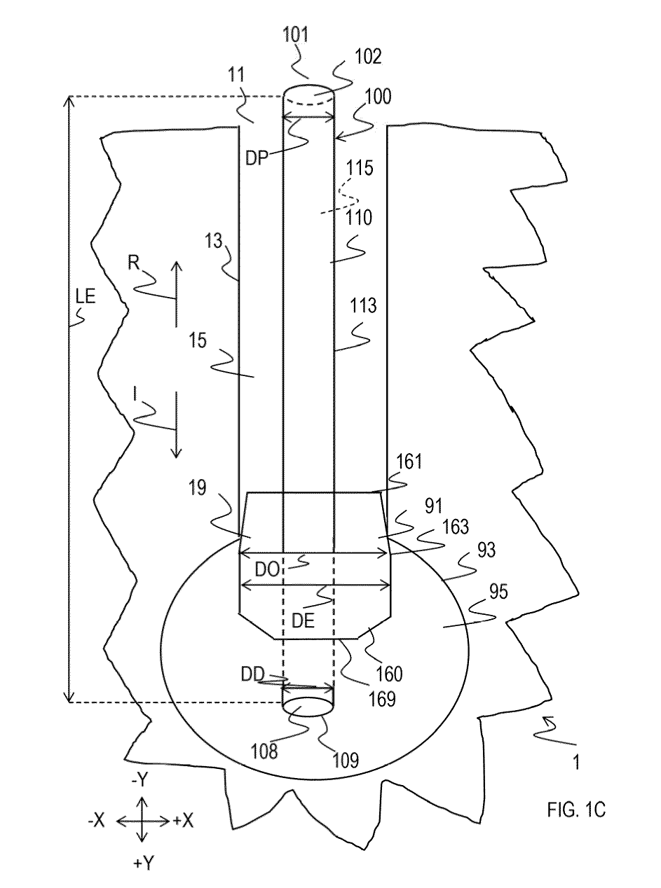

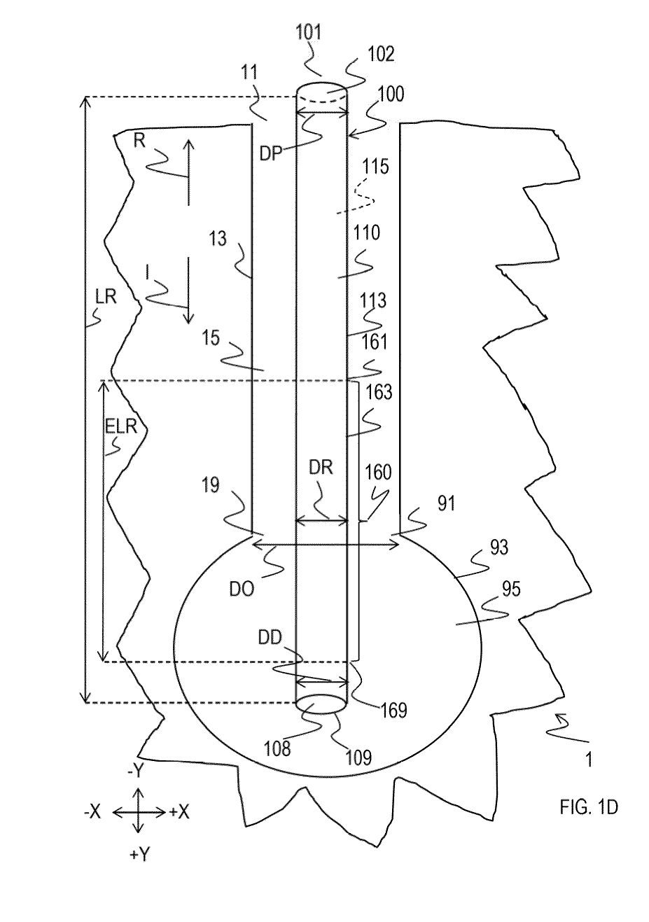

FIGS. 1-1D show an illustrative assembly 100 in various configurations or stages of use with respect to a patient 1. Assembly 100 may be an intubation assembly or any other suitable assembly for use in any suitable procedure with respect to any suitable patient 1. As shown in FIGS. 1-1D, for example, assembly 100 may extend between a proximal or first assembly end 101, which may have an outer cross-sectional dimension (e.g., diameter) DP, and a distal or second assembly end 109, which may have an outer cross-sectional dimension (e.g., diameter) DD. Assembly 100 may include at least one tube or tube subassembly 110 that may extend between ends 101 and 109. Tube subassembly 110 may include at least one tube wall 113 that may define at least one internal passageway 115 extending along at least a portion of assembly 100. Wall 113 may also include at least one proximal or first tube opening 102 that may provide access to passageway 115 at or near end 101 of assembly 100 and at least one distal or second tube opening 108 that may provide access to passageway 115 at or near end 109 of assembly 100. Moreover, assembly 100 may also include an expander or expander subassembly 160 that may extend along at least a portion of tube subassembly 110, where expander subassembly 160 may include an external surface 163. As also shown in FIGS. 1-1D, for example, patient 1 may include a passageway wall 13 that may define a passageway 15 that may extend between at least one proximal or first access opening 11 and a distal or second opening 19. Moreover, patient 1 may include a target wall 93 that may define at least a portion of a target space 95, where a proximal or first target opening 91 of wall 93 may be coupled to opening 19 of passageway 15, such that passageway 15 may be fluidly coupled to target space 95. As shown in FIGS. 1-1D, for example, at least a portion of passageway 15 and/or the coupling of opening 19 and opening 91 may have a cross-sectional dimension (e.g., diameter) DO, which may be a minimum dimension of patient 1 through which at least a portion of assembly 100 may pass or otherwise exist during any stage of use within patient 1.

When in an insertion state (see, e.g., FIG. 1), assembly 100 may be inserted into patient 1 to a particular position, and then assembly 100 may be re-configured into an expanded state (see, e.g., FIG. 1A and/or FIG. 1B and/or FIG. 1C) within patient 1 such that assembly 100 may be safely used within patient 1. After use of assembly 100 in its expanded state within patient 1, assembly 100 may be re-configured into a removal state (see, e.g., FIG. 1D) within patient 1 for removal of assembly 100 from patient 1. For example, as shown by FIG. 1, assembly 100 may first be configured in an insertion state or configuration such that assembly 100 may then be at least partially inserted into patient 1. In some embodiments, end 109 of assembly 100 in its insertion state may be inserted into patient 1 in the direction of arrow I through opening 11, through passageway 15, through opening 19, through opening 91, and into target space 95, such that at least one opening 108 of assembly 100 may be within space 95 and/or such that at least one opening 102 of assembly 100 may be accessible to an operator O of assembly 100 (e.g., a physician or patient 1 itself), who may be external to patient 1. Assembly 100 may be of a length LI that may extend between end 101 and end 109 of assembly 100 in its insertion state, and where such a length provided by assembly 100 in its insertion state may vary based on the size of patient 1 and the procedure to be performed. As shown in FIG. 1, when assembly 100 is in its insertion state, no portion of expander 160 may have a cross-sectional dimension (e.g., diameter) greater than dimension DI. In some embodiments, dimension DD of end 109 and dimension DI of expander 160 in the insertion state of assembly 100 may be less than dimension DO of patient 1 such that assembly 100 in its insertion state may be safely inserted into patient 1 without damaging wall 13 and/or wall 93 of patient 1.

After assembly 100 has been inserted into patient 1 while assembly 100 is in its insertion state, assembly 100 may be re-configured into an expanded state within patient 1 such that assembly 100 may thereafter be safely used within patient 1. For example, as shown in each one of FIGS. 1A-1C, once assembly 100 in its insertion state has been inserted into its insertion position of FIG. 1 within patient 1, assembly 100 may be re-configured into an expanded state within patient 1 such that assembly 100 may thereafter be safely used in that expanded state within patient 1. As shown in each one of FIGS. 1A-1C, when assembly 100 is in its expanded state, at least a portion of expander 160 may have a maximum cross-sectional dimension (e.g., diameter) DE that may be at least equal to or greater than dimension DO of patient 1, such that at least a portion of wall 163 of expander 160 may contact or otherwise interact with at least a portion of wall 93 of target 95 and/or with at least a portion of wall 13 of passageway 15 for safely securing expanded assembly 100 at a particular position within patient 1 and/or for safely preventing certain material from traveling between wall 163 of expander 160 and at least a portion of wall 93 of target 95 and/or at least a portion of wall 13 of passageway 15. One or more of dimensions DE, DI, and DR may be widths defined by expander 160, where such a width may be perpendicular to the length of expander 160 (e.g., along the X-axis, which may be perpendicular to the length extending between ends 161 and 169 of expander 160 along the Y-axis). As shown in FIG. 1A, for example, all of expander 160 may be positioned within target space 95 when assembly 100 is re-configured from its insertion state into its expanded state, such that at least a portion of wall 163 of expander 160 may contact or otherwise interact with at least a portion of wall 93 of target 95. Alternatively, as shown in FIG. 1B, for example, all of expander 160 may be positioned within passageway 15 when assembly 100 is re-configured from its insertion state into its expanded state, such that at least a portion of wall 163 of expander 160 may contact or otherwise interact with at least a portion of wall 13 of passageway 15. Alternatively, as shown in FIG. 1C, for example, a first portion of expander 160 may be positioned within passageway 15 and a second portion of expander 160 may be positioned with target space 95 when assembly 100 is re-configured from its insertion state into its expanded state, such that at least a first portion of wall 163 of expander 160 may contact or otherwise interact with at least a portion of wall 13 of passageway 15 and such that at least a second portion of wall 163 of expander 160 may contact or otherwise interact with at least a portion of wall 93 of target 95. As shown in FIGS. 1A-1C, at least a portion of expander 160 may expand at least along the X-axis such that a maximum cross-sectional dimension (e.g., diameter) of expander 160 may expand from dimension DI to dimension DE when assembly 100 is reconfigured from its insertion state to its expanded state. As shown in FIGS. 1A-1C, assembly 100 may be of a length LE that may extend between end 101 and end 109 of assembly 100 in its expanded state, where such a length LE provided by assembly 100 may vary based on the size of patient 1 and may be greater than, less than, or equal to length LI of the insertion state and/or length LR of the removal state (described below).

Once assembly 100 has been expanded into its expanded state within patient 1 (e.g., as shown in any one or more of FIGS. 1A-1C), assembly 100 may be safely used within patient 1 in any suitable way, such as in any suitable intubation process. For example, in some embodiments, expanded assembly 100 may be safely used within patient 1 for injecting material (e.g., treatment material, such as nutrients or medicine or oxygen) through opening 102, into and through passageway 115, then out of passageway 115 through opening 108, and into target space 95 of patient 1, and/or for removing material (e.g., treatment material, such as waste) from target space 95, through opening 108, into and through passageway 115, then out of passageway 115 through opening 102 away from patient 1. In certain embodiments, target space 95 may be a stomach, opening 91 may be a lower esophageal sphincter, passageway 15 may be an esophagus, pharynx, throat, and/or nasal cavity, and opening 11 may be a nostril or mouth of patient 1, where assembly 100 may be used during a nasogastric intubation process. In other embodiments, target space 95 may be a bladder, opening 91 may be a sphincter, passageway 15 may be a urethra, and opening 11 may be a urinary meatus of patient 1 where assembly 100 may be used during any suitable process that might otherwise use a Foley catheter. It is to be understood that assembly 100 may be used with respect to any suitable portions of any suitable patient 1 for any suitable process, where expander 160 may be expanded such that at least a portion of wall 163 of expander 160 may contact or otherwise interact with at least a portion of wall 93 of target 95 and/or with at least a portion of wall 13 of passageway 15 for safely securing expanded assembly 100 at a particular position within patient 1 (e.g., for preventing end 109 of assembly 100 from being inadvertently removed from target space 95 (e.g., in the direction of arrow R), such as when assembly 100 may be used as a Foley catheter) and/or for safely preventing certain material from traveling between wall 163 of expander 160 and at least a portion of wall 93 of target 95 and/or between wall 163 of expander 160 and at least a portion of wall 13 of passageway 15 (e.g., for preventing contents of a stomach target 95 from escaping target 95 through passageway 15 about the exterior of wall 163 of expander 160 (i.e., not through assembly 100), such as towards a trachea or other portion of patient 1 between expander 160 and end 11 of passageway 15 that may cause infections (e.g., in the direction of arrow R), such as when assembly 100 may be used as a nasogastric tube (e.g., a Levin catheter, a Salem Sump catheter, a Dobhoff tube, etc.)). Specifically, reflux of contents from the stomach back into the esophagus has been a persistent problem, especially in the presence of nasogastric tubes. Contents often attempt to travel back up from the stomach around the tube, thereby causing reflux esophagitis, aspiration pneumonitis, and/or pneumonias.

After assembly 100 has been used in its expanded state within patient 1, assembly 100 may be re-configured into a removal state such that assembly 100 may thereafter be safely removed from within patient 1 (e.g., in the direction of arrow R). For example, as shown in FIG. 1D, once assembly 100 has been used in its expanded state of any of FIGS. 1A-1C within patient 1, assembly 100 may be re-configured into a removal state within patient 1 such that assembly 100 may thereafter be safely removed in its removal state from within patient 1. For example, as shown in FIG. 1D, when assembly 100 is in its removal state, no portion of expander 160 may have a cross-sectional dimension (e.g., diameter) greater than dimension DR, where such a dimension DR provided by assembly 100 may vary based on the size of patient 1 and may be greater than, less than, or equal to dimension DI of the insertion state. In some embodiments, dimension DD of end 109 and dimension DR of expander 160 in the removal state of assembly 100 may be less than dimension DO of patient 1 such that assembly 100 in its removal state may be safely removed from patient 1 without damaging wall 13 and/or wall 93 of patient 1. In some embodiments, as shown in FIG. 1D, at least a portion of expander 160 may contract at least along the X-axis such that a maximum cross-sectional dimension (e.g., diameter) of expander 160 may contract from dimension DE to dimension DR when assembly 100 is reconfigured from its expanded state to its removal state. As shown in FIG. 1D, assembly 100 may be of a length LR that may extend between end 101 and end 109 of assembly 100 in its removal state, where such a length LR provided by assembly 100 may vary based on the size of patient 1 and may be greater than, less than, or equal to length LI of the insertion state and/or length LE of the expanded state.

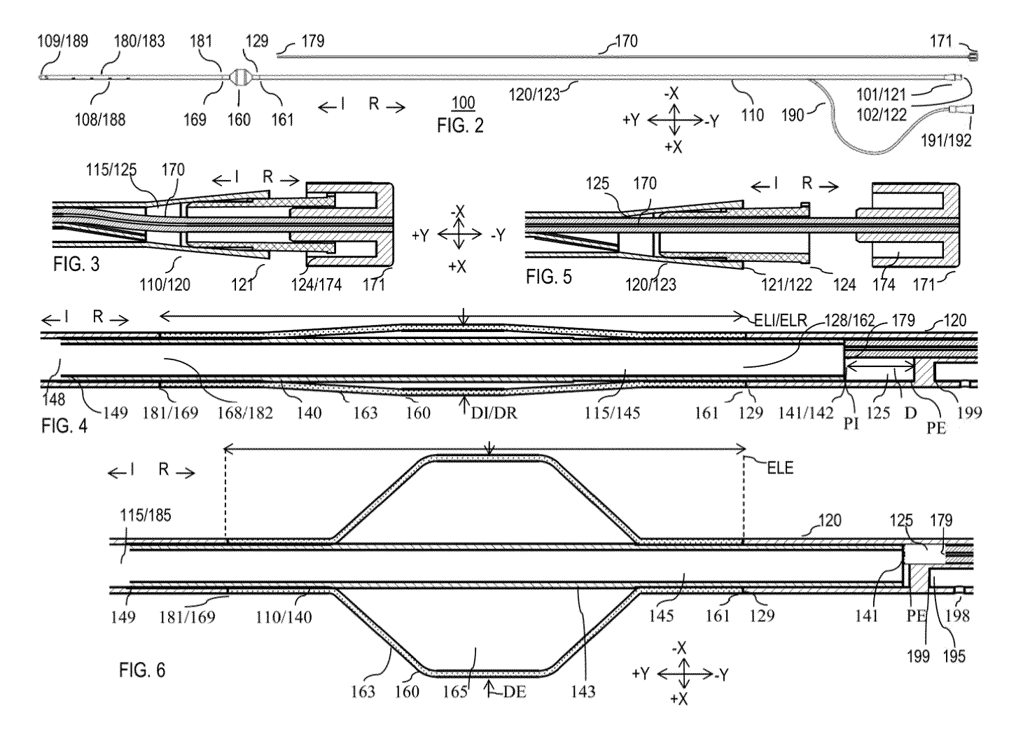

In some embodiments, expander subassembly 160 may include a balloon or other mechanism that may be inflatable by air or other suitable fluid for enabling the expansion of at least a portion of expander subassembly 160 (e.g., from dimension DI to dimension DE), which may allow at least a portion of expander 160 to contact a wall of patient 1 for securing expanded assembly 100 at a particular position within patient 1 and/or for preventing certain material from traveling between expander 160 and a wall of patient 1. However, such an inflatable balloon expander may be dangerous as it may be difficult to finely control the amount by which the balloon is inflated during use within patient 1. As the natural or relaxed state of a balloon expander may be in its un-inflated state (e.g., when no external forces of the assembly are being applied to the balloon), such a balloon expander must be reconfigured into its unnatural or tensioned inflated state within patient 1 for expanded use of the intubation assembly, where the dimensions and other characteristics of such an unnatural expanded state may be difficult to control or predict within patient 1. For example, over-inflation of a balloon may cause such a balloon expander to compress an interior wall of patient 1 (e.g., wall 13 and/or 93), which may cause blood flow to stop or other dangerous effects (e.g., necrosis). As another example, over-inflation of a balloon may cause such a balloon expander to provide damaging pressure against an interior wall of a patient or may cause the balloon to explode or pop, any of which may damage an interior wall of patient 1 (e.g., esophageal rupture or esophageal necrosis). Moreover, a dimension of at least a portion of patient 1 may vary during use of assembly 100. For example, cross-sectional dimension DO of passageway 15 and/or target 95 may expand and contract while assembly 100 is positioned within patient 1, such as due to patient 1 breathing or swallowing. Such patient expansion and contraction may pop or rupture a balloon expander. Moreover, repeated inflation and deflation of such a balloon expander during re-configuration between insertion, expansion, and removal states of assembly 100 may cause such a balloon to lose its elasticity over time, thereby diminishing the value of assembly 100. Therefore, various other embodiments for expander subassembly 160 are described below, such as with respect to FIGS. 2-17, that may increase the safety with which assembly 100 may be expanded and used within patient 1. It is to be noted that, while “proximal” or “proximate” may be used herein to refer to a general direction or end of assembly 100 that may be closest to operator O of assembly 100 during use (e.g., external to patient 1), and while “distal” or “distant” may be used herein to refer to a general direction or end of assembly 100 that may be farthest from operator O of assembly 100 during use (e.g., within target 95), such directional and orientational terms may be used herein only for convenience, and that no fixed or absolute directional or orientational limitations are intended by the use of these words.

FIGS. 2-6D show an illustrative assembly 100 in different configurations or stages of use for any suitable procedure with respect to patient 1 of FIGS. 1-1D. As shown in FIGS. 2-6D, in some embodiments, assembly 100 may include a first or proximal tube 120, a deployment tube 140, an expander 160, a deployment mechanism 170, and a second or distal tube 180. For example, proximal tube 120 may extend between a proximal or first end 121 (e.g., assembly end 101) and a distal or second end 129. Proximal tube 120 may include at least one tube wall 123 that may define at least one internal passageway 125 (e.g., at least a portion of passageway 115) extending along at least a portion of assembly 100. Wall 123 may also include at least one proximal or first tube opening 122 (e.g., opening 102) that may provide access to passageway 115/125 at or near end 101/121 of assembly 100 and at least one distal or second tube opening 128 that may provide access to passageway 125 at or near end 129 of proximal tube 120. Distal tube 180 may extend between a proximal or first end 181 and a distal or second end 189 (e.g., assembly end 109). Distal tube 180 may include at least one tube wall 183 that may define at least one internal passageway 185 (e.g., at least a portion of passageway 115) extending along at least a portion of assembly 100. Wall 183 may also include at least one proximal or first tube opening 182 that may provide access to passageway 115/185 at or near end 181 of distal tube 180 and at least one distal or second tube opening 188 (e.g., opening 108) that may provide access to passageway 115/185 at or near end 189 of distal tube 180. As shown, expander 160 may include a wall defining an external surface 163 and an expander passageway 165 (e.g., at least a portion of passageway 115) that may extend between a first or proximal expander end 161 and a second or distal expander end 169. A wall defining external surface 163 may also include at least one proximal or first expander opening 162 that may provide access to passageway 165 at or near end 161 of expander 160 and at least one distal or second expander opening 168 that may provide access to passageway 165 at or near end 169 of expander 160. Deployment mechanism 170 may extend between a first or proximal end 171 and a second or distal end 179. Moreover, deployment tube 140 may extend between a proximal or first end 141 and a distal or second end 149. Deployment tube 140 may include at least one tube wall 143 that may define at least one internal passageway 145 (e.g., at least a portion of passageway 115) extending along at least a portion of assembly 100. Wall 143 may also include at least one proximal or first tube opening 142 that may provide access to passageway 115/145 at or near end 141 of deployment tube 140 and at least one distal or second tube opening 148 that may provide access to passageway 115/145 at or near end 149 of deployment tube 140.

Expander 160 may be fluidly coupled in any suitable way to both proximal tube 120 and distal tube 180 (e.g., such that passageway 115 may include passageways 125, 165, and 185). For example, as shown in FIGS. 2, 4, and 6A-6D, expander 160 may be coupled at or near proximal end 161 to proximal tube 120 at or near distal end 129 such that openings 162 and 128 may be fluidly coupled, while expander 160 may be coupled at or near distal end 169 to distal tube 180 at or near proximal end 181 such that openings 168 and 182 may be fluidly coupled. As described below in more detail, expander 160 may be operative to be reconfigured between a first natural or relaxed state (e.g., when no external forces of assembly 100 are being applied to expander 160) and a second unnatural or tensioned state (e.g., when one or more external forces may be applied by other portions of assembly 100 on expander 160). For example, as shown in FIGS. 1A, 2, and 6A-6D, expander 160 may be in a natural or relaxed state when ends 161 and 169 may be allowed to retract towards each other such that a length ELE may separate ends 161 and 169, whereby at least a portion of expander 160 may have a maximum cross-sectional dimension (e.g., diameter) DE, which may be at least equal to or greater than dimension DO of patient 1 (e.g., as described above, such that at least a portion of wall 163 of expander 160 may contact or otherwise interact with at least a portion of a wall of patient 1 for safely securing expanded assembly 100 at a particular position within patient 1 and/or for safely preventing certain material from traveling between wall 163 of expander 160 and at least a portion of a wall of target 95 and/or passageway 15 of patient 1), such that such a natural or relaxed state of expander 160 may be used for an expanded state of assembly 100 within patient 1. However, as shown in FIGS. 1, 1D, 4, and 4A, for example, expander 160 may be in an unnatural or tensioned state when ends 161 and 169 may be forced away from each other such that a length ELI and/or ELR may separate ends 161 and 169, whereby no portion of expander 160 may have a cross-sectional dimension (e.g., diameter) greater than dimension DI and/or DR, which may be less than dimension DO of patient 1 (e.g., as described above), such that such an unnatural or tensioned state of expander 160 may be used for an insertion state into patient 1 and/or a removal state of assembly 100 from within patient 1. Assembly 100 may be provided with any suitable components or features for reconfiguring expander 160 between its natural and un-natural states (e.g., between the expanded and insertion/removal states of assembly 100).

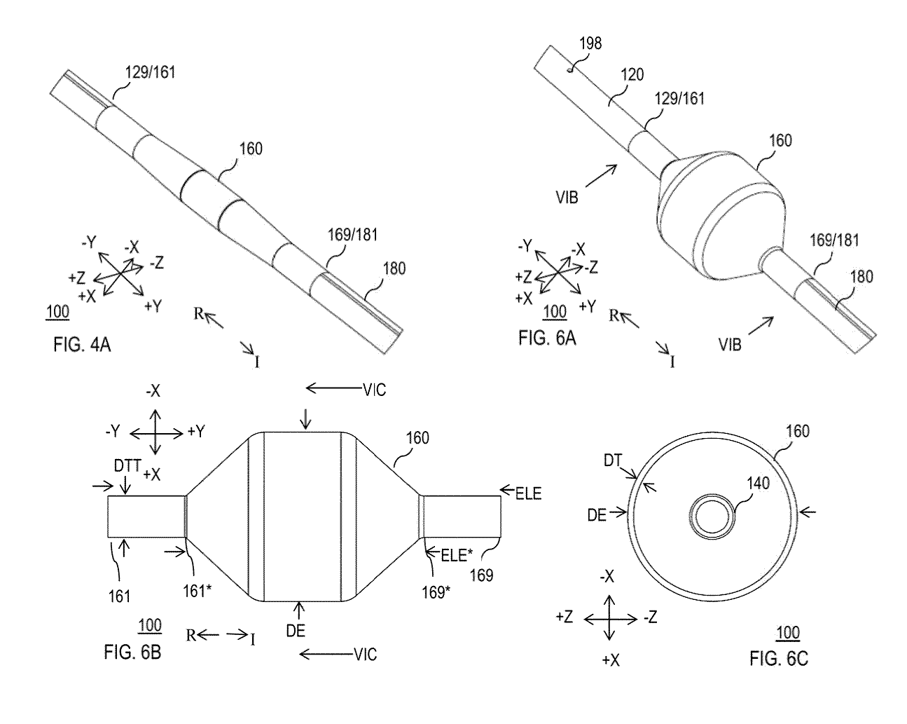

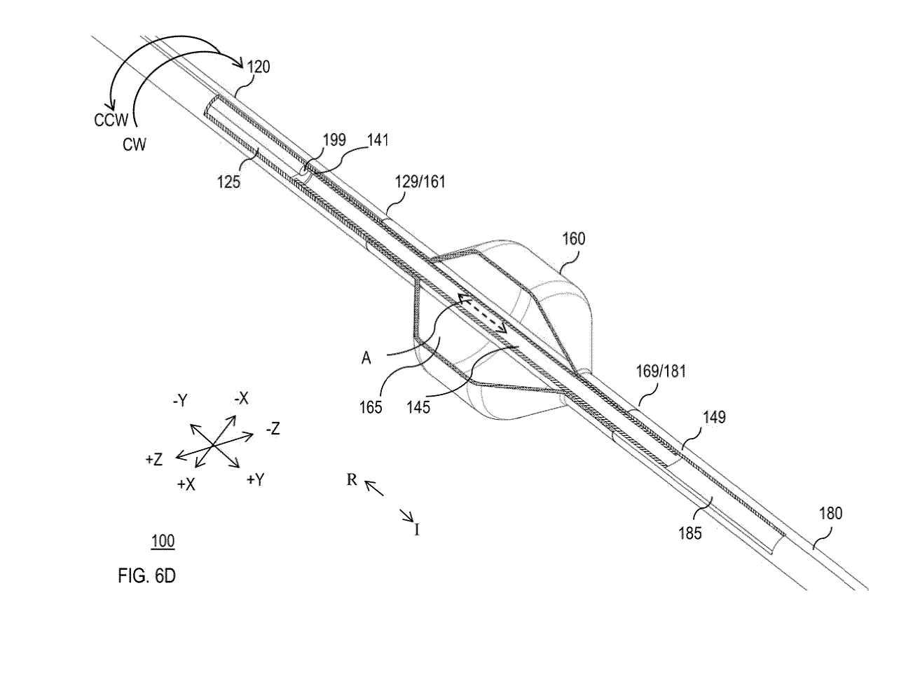

In some embodiments, deployment tube 140 may be positioned within assembly 100 such that deployment mechanism 170 may be configured to move deployment tube 140 along assembly 100 for adjusting the distance between ends 161 and 169 of expander 160, thereby reconfiguring expander 160 between its natural and un-natural states (e.g., thereby reconfiguring assembly 100 between its expanded state and insertion/removal state). In such embodiments, passageway 145 of deployment tube 140 may be fluidly coupled with passageway 125 of proximal tube 120 and passageway 185 of distal tube 180 in any suitable way (e.g., such that passageway 115 may include passageways 125, 145, and 185 and/or such that passageway 145 may be provided through passageway 165). For example, as shown in FIGS. 4 and 6A-6D, opening 142 and proximal end 141 of deployment tube 140 may be positioned within passageway 125 of proximal tube 120, while opening 148 and distal end 149 of deployment tube 140 may be positioned within passageway 185 of distal tube 180, and/or while at least a portion of passageway 125 may extend through at least a portion of passageway 165 of expander 160. Alternatively, in other embodiments, opening 128 and distal end 129 of proximal tube 120 may be positioned within passageway 145 of deployment tube 140 and/or opening 182 and proximal end 181 of distal tube 180 may be positioned within passageway 145 of deployment tube 140. Alternatively, in other embodiments, proximal end 181 of distal tube 180 may be coupled to distal end 149 of deployment tube 140 such that openings 182 and 148 may be fluidly coupled. In some embodiments, when in the expanded state of FIGS. 1A and 6A-6D, assembly 100 may be operative to communicate material between opening 101/121 and opening 109/189 through at least a portion of passageway 125, at least a portion of passageway 145, and at least a portion of passageway 185.

Such that movement of deployment tube 140 along the length of assembly 100 (e.g., along the Y-axis) may adjust the distance between ends 161 and 169 of expander 160, a portion of deployment tube 140 (e.g., at or near distal end 149) may be coupled to a portion of expander 160 (e.g., at or near distal end 169) and/or to at least a portion of distal tube 180 (e.g., at or near proximal end 181). For example, in some embodiments, at least a portion of expander 160 (e.g., at or near distal end 169) may be attached or otherwise coupled to a portion of deployment tube 140 (e.g., at or near distal end 149), while at least a portion of distal tube 180 (e.g., at or near proximal end 181) may also be attached or otherwise coupled to a portion of deployment tube 140 (e.g., at or near distal end 149), such that expander 160 may or may not be coupled to distal tube 180 except via deployment tube 140. In other embodiments, at least a portion of expander 160 (e.g., at or near distal end 169) may be attached or otherwise coupled to a portion of distal tube 180 (e.g., at or near proximal end 181), while a portion of expander 160 (e.g., at or near distal end 169) may also be attached or otherwise coupled to a portion of deployment tube 140 (e.g., at or near distal end 149), such that deployment tube 140 may or may not be coupled to distal tube 180 except via expander 160. In yet other embodiments, at least a portion of distal tube 180 (e.g., at or near proximal end 181) may be attached or otherwise coupled to a portion of expander 160 (e.g., at or near distal end 169), while a portion of distal tube 180 (e.g., at or near proximal end 181) may also be attached or otherwise coupled to a portion of deployment tube 140 (e.g., at or near distal end 149), such that deployment tube 140 may or may not be coupled to expander 160 except via distal tube 180.

While a distal portion of deployment tube 140 (e.g., at or near distal end 149) may be coupled to a distal portion of expander 160 (e.g., at or near distal end 169) and/or to a portion of distal tube 180 (e.g., at or near proximal end 181), deployment tube 140 (e.g., at least a proximal portion of deployment tube 140 at or near proximal end 141) may be configured to move along assembly 100 with respect to another portion of expander 160 (e.g., at or near proximal end 161) and/or with respect to proximal tube 120, such that movement of deployment tube 140 along the length of assembly 100 (e.g., along the Y-axis) may adjust the distance between ends 161 and 169 of expander 160. For example, as shown in FIGS. 4 and 6, at least a portion of deployment tube 140 may be positioned within passageway 125 of proximal tube 120 such that deployment tube 140 may move with respect to proximal tube 120 within passageway 125 (e.g., along the Y-axis). For example, deployment tube 140 may be configured to move with respect to proximal tube 120 from a first position of FIG. 6, where proximal end 141 of deployment tube 140 may be at or adjacent a point PE along passageway 115 and where ends 161 and 169 of expander 160 may be separated by a distance ELE such that expander 160 may be in its relaxed and natural expanded state of FIGS. 1A and 6, to a second position of FIG. 4, where proximal end 141 of deployment tube 140 may be at a point PI that may be spaced by a distance D distally (e.g., in the +Y direction) from point PE along passageway 115 and where ends 161 and 169 of expander 160 may be separated by a distance ELI or ELR such that expander 160 may be in its tensioned and unnatural insertion or removal state of FIG. 1 or 1D and 4. Following this example, as also shown in FIGS. 3-6, deployment mechanism 170 may be utilized to enable such movement of deployment tube 140 and, thus, expander 160. For example, as shown in FIGS. 3 and 4, distal end 179 of deployment mechanism 170 may be fully inserted into passageway 125 of proximal tube 120 (e.g., in +Y direction along the Y-axis via opening 102/122 at end 101/121 of proximal tube 120 of subassembly 110), such that distal end 179 of deployment mechanism 170 may push proximal end 141 of deployment tube 140 to point PI at a distance D beyond point PE, and such that deployment mechanism 170 may hold deployment tube in that position for maintaining expander 160 and assembly 100 in its insertion/removal state until assembly 100 is safely inserted into patient 1 or safely removed from patient 1. For example, in some embodiments, operator O may insert deployment mechanism 170 into subassembly 110 and hold deployment mechanism 170 at the position of FIGS. 3 and 4 for maintaining assembly 100 in its insertion/removal state (e.g., by providing an insertion force on end 171 in the +Y direction). Alternatively, a portion of deployment mechanism 170 and a portion of subassembly 110 may interact with each other for holding deployment mechanism 170 at the position of FIGS. 3 and 4. For example, as shown in FIGS. 2 and 3, deployment mechanism 170 may include a retention feature 174 (e.g., at or near end 171) that may interact in any suitable way with a retention feature 124 of subassembly 110 (e.g., at or near end 121 of proximal tube 120) for maintaining the relationship of FIGS. 3 and 4 between deployment mechanism 170, deployment tube 140, and expander 160 (e.g., for maintaining expander 160 and assembly 100 in its insertion/removal state until assembly 100 is safely inserted into patient 1 or safely removed from patient 1). For example, in some embodiments, retention feature 174 may include one or more threads with which one or more screw elements of retention feature 124 may interact with, or vice versa, or retention features 124/174 may enable a tight fit therebetween.

It is to be understood that any force that may be exerted by expander 160 (e.g., in the −Y direction) on deployment mechanism 170 (e.g., via deployment tube 140 (e.g., via distal tube 180)) due to expander 160 being configured to return to its natural relaxed state (e.g., of FIGS. 5 and 6, where ends 161 and 169 are separated by a distance ELE that may be shorter than distance ELI/ELR of FIG. 4) may be overcome by a force exerted by deployment mechanism 170 on expander 160 (e.g., via deployment tube 140 (e.g., via distal tube 180)) due to operator O or retention features 124/174 maintaining the assembly state of FIGS. 3 and 4. However, when assembly 100 in such an insertion assembly state is properly positioned within patient 1 (e.g., as shown in FIG. 1), deployment mechanism 170 may be allowed to move proximally (e.g., in the −Y direction, by at least distance D from point PI to point PE), such that expander 160 may reconfigure itself to its natural and relaxed configuration of FIGS. 1A and 6, whereby expander 160 may retract its ends 161 and 169 closer to each other (e.g., to a distance ELE). Such relaxation of expander 160 may be possible due to proximal end 141 of deployment tube 140 being able to move proximally from point PI to point PE (e.g., due to no deployment force being exerted on deployment tube 140 by deployment mechanism 170 (e.g., until at least point PE)). That is, when operator O maintains proximal end 121 of proximal tube 120 at a particular position (e.g., at or near opening 11 of patient 1) after discontinuing a particular deployment force from being exerted on deployment tube 140 by deployment mechanism 170, any force that may be exerted by expander 160 due to expander 160 being configured to return to from its unnatural tensioned state to its natural relaxed state (e.g., for reducing the distance between ends 161 and 169 from distance ELI/ELR of FIG. 4 to distance ELE of FIG. 5) may pull distal end 169 of expander 160 and, thus, portions of distal tube 180 and deployment tube 140 towards proximal end 161 of expander 160 (e.g., in the −Y direction), which may thereby pull proximal end 141 of deployment tube 140 distance D from point PI to point PE (e.g., in the −Y direction), where distance D may be equal to the difference between distance ELI and distance ELE. In some embodiments, deployment mechanism 170 may be completely removed from pathway 115 of subassembly 110 (e.g., pathway 125 of proximal tube 120) when expander 160 is in its expanded natural state such a maximum amount of pathway 115 may be utilized for injecting fluid into patient 1 and/or for removing fluid from patient 1 (e.g., as shown in FIG. 2). Alternatively, deployment mechanism 170 may remain within at least a portion of subassembly 110 during safe use of assembly 100 within patient 1 once distal end 179 has been moved proximally to at least point PE, such that expander 160 may be able to fully reconfigure to its natural expanded state (e.g., as shown in FIGS. 5 and 6).

In some embodiments, as shown in FIGS. 2-6, assembly 100 may also include a supplemental tube subassembly 190 that may be provided to treat (e.g., extract material from and/or inject material into) a supplemental region of patient 1 that may be proximal to target 95 and proximal to expander 160 when assembly 100 is in its expanded state within patient 1. For example, as shown, subassembly 190 may include a tube defining a passageway 195 that may extend from a proximal end 191 to a distal end 199. A proximal opening 192 for passageway 195 may be provided at or near proximal end 191 and a distal opening 198 for passageway 195 may be provided at or near distal end 199. Fluid may be injected into patient 1 through passageway 195 from opening 192 to opening 198 and/or fluid may be removed from patient 1 through passageway 195 from opening 198 to opening 192. As shown, at least a portion of passageway 195 may be provided within passageway 115 (e.g., within passageway 125 of proximal tube 120). In such embodiments, distal end 199 of subassembly 190 may be positioned at point PE, such that when distal end 179 of deployment mechanism 170 has been removed from subassembly 110 at least proximally beyond point PE, distal end 199 of subassembly 190 may be configured to prevent deployment tube 140 from moving proximally beyond point PE, which may prevent ends 161 and 169 of expander 160 from retracting towards each other to a separation distance less than distance ELE. In other embodiments, neither distal end 179 of deployment mechanism 170 nor distal end 199 of subassembly 190 nor any other portion of any other component of assembly 100 may be positioned at point PE for preventing proximal movement of deployment tube 140 (e.g., in the −Y direction). Instead, expander 160 may be configured to prevent ends 161 and 169 of expander 160 from retracting towards each other to a separation distance less than distance ELE.

In some embodiments, rather than altering the distance between ends 161 and 169 for reconfiguring expander 160 between its expanded natural state and its deformed unnatural state, one of ends 161 and 169 may be rotated with respect to the other one of ends 161 and 169 for reconfiguring expander 160. For example, as shown in FIG. 6D, a first one of ends 161 and 169 of expander 160 may be rotated with respect to a second one of ends 161 and 169 of expander 160 in either a clockwise direction of arrow CW or a counterclockwise direction of arrow CCW about a longitudinal axis A (e.g., an axis of assembly 100 or of expander 160, such as along the Y-axis). Such rotation may or may not affect the distance ELE between ends 161 and 169 but may reduce the cross-sectional dimension of expander 160 from DE to DI or DR.

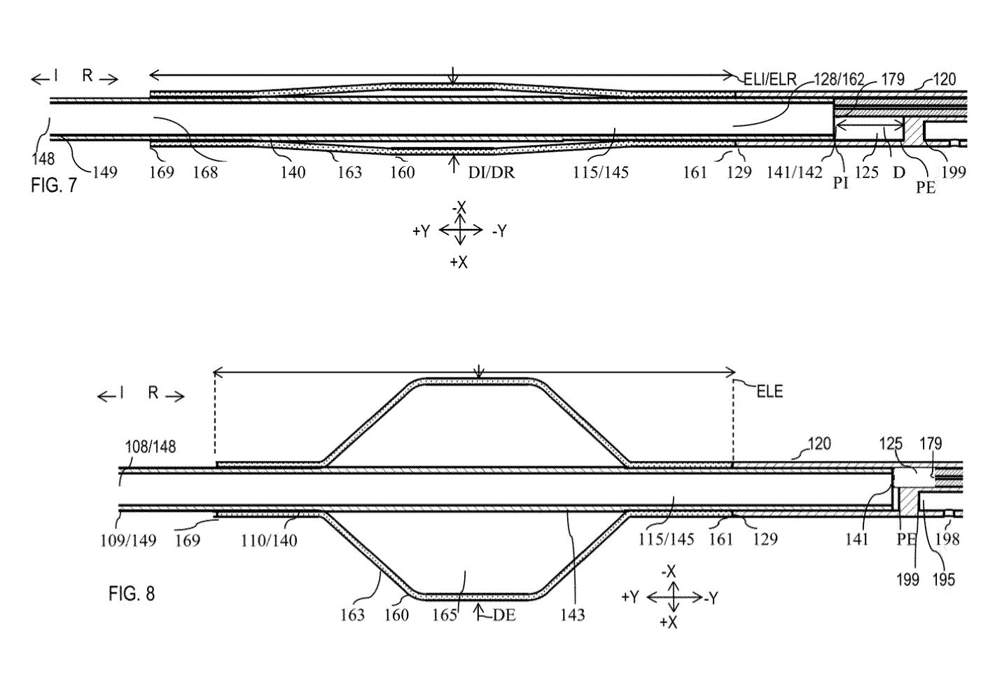

In some embodiments, assembly 100 may not include a distal tube 180. For example, as shown in FIGS. 7 and 8, rather than including a distal tube (e.g., distal tube 180 of FIGS. 2-6D), a portion of deployment tube 140 (e.g., at or near distal end 149) may be coupled to a portion of expander 160 (e.g., at or near distal end 169), while one or more openings 148 at or near distal end 149 of deployment tube 140 may act as opening 108 of assembly 100 (e.g., for passing material from patient 1 into passageway 115 and/or from passageway 115 into patient 1). Otherwise, assembly 100 of FIGS. 7 and 8 may act similarly to assembly 100 of FIGS. 2-6D, as described above.

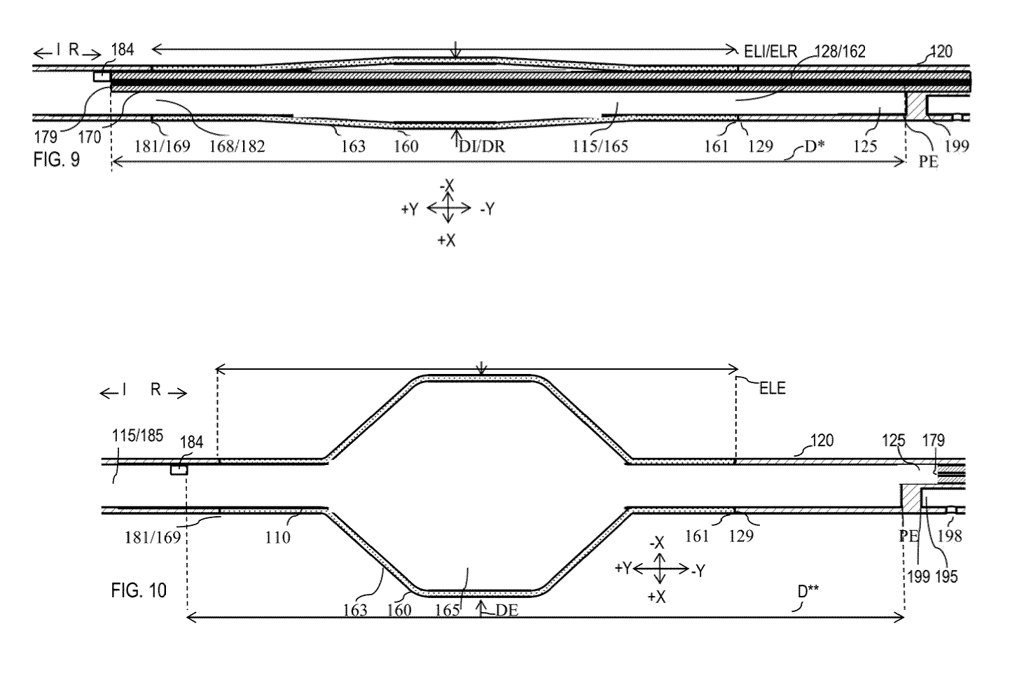

In some embodiments, assembly 100 may not include a deployment tube 140. For example, as shown in FIGS. 9 and 10, rather than including a deployment tube (e.g., deployment tube 140 of FIGS. 2-6D), a portion of subassembly 110 (e.g., at or near distal end 169 of expander 160 and/or at or near proximal end 181 of distal tube 180 and/or elsewhere along distal tube 180) may include a deployment feature 184 (e.g., within passageway 115). As shown in FIG. 9, for example, deployment feature 184 may be configured to receive a deployment force provided by distal end 179 of deployment mechanism 170 (e.g., in the +Y direction), which may force deployment feature 184 a distance D* away from point PE, and, as shown in FIG. 10, when such a deployment force is terminated (e.g., by withdrawing deployment mechanism proximally), expander 160 may pull deployment feature 184 proximally towards point PE to within a shorter distance D**. That is, when operator O maintains proximal end 121 of proximal tube 120 at a particular position (e.g., at or near opening 11 of patient 1) after discontinuing a particular deployment force from being exerted on deployment feature 184 by deployment mechanism 170, any force that may be exerted by expander 160 due to expander 160 being configured to return to from its unnatural tensioned state to its natural relaxed state (e.g., for reducing the distance between ends 161 and 169 from distance ELI/ELR of FIG. 9 to distance ELE of FIG. 10) may pull distal end 169 of expander 160 and, thus, portions of distal tube 180 towards proximal end 161 of expander 160 (e.g., in the −Y direction), which may thereby pull deployment feature 184 towards point PE by a distance that is the difference between D* and D** (e.g., in the −Y direction), where the difference between distance D* and distance D** may be equal to the difference between distance ELI and distance ELE. Otherwise, assembly 100 of FIGS. 9 and 10 may act similarly to assembly 100 of FIGS. 2-6D, as described above.

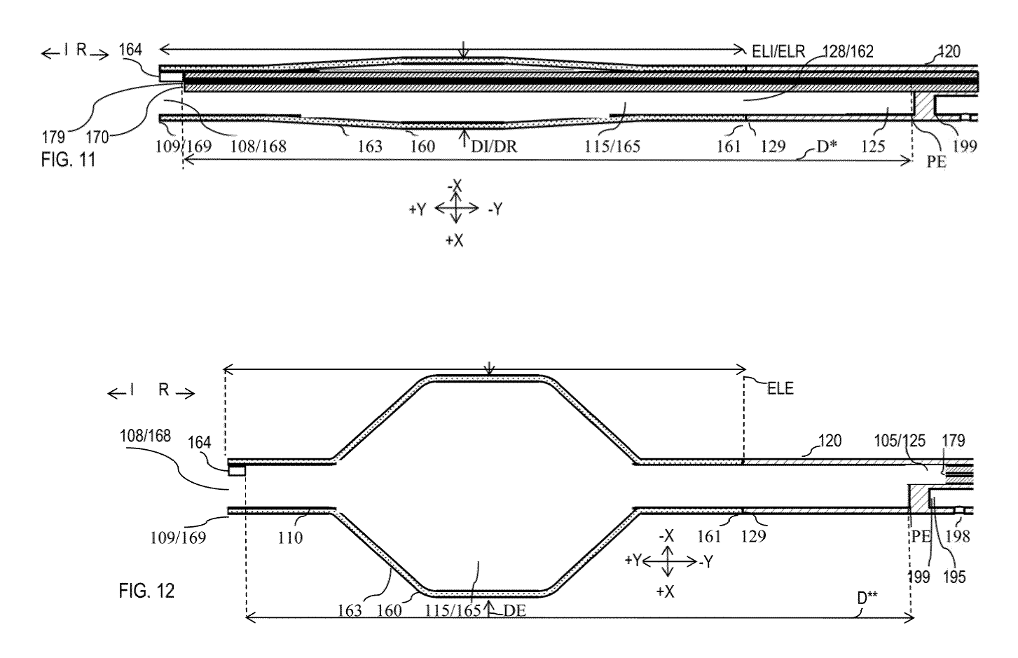

In yet some embodiments, assembly 100 may not include a deployment tube 140 or a distal tube 180. For example, as shown in FIGS. 11 and 12, rather than including a deployment tube and a distal tube (e.g., deployment tube 140 and distal tube 180 of FIGS. 2-6D), a portion of expander 160 (e.g., at or near distal end 169 of expander 160) may include a deployment feature 164 (e.g., within passageway 115), for example, as described above with respect to FIGS. 9 and 10, while one or more openings 168 at or near distal end 169 of expander 160 may act as opening 108 of assembly 100 (e.g., for passing material from patient 1 into passageway 115 and/or from passageway 115 into patient 1). Otherwise, assembly 100 of FIGS. 11 and 12 may act similarly to assembly 100 of FIGS. 2-6D, as described above.

Unlike a balloon, expander 160 may be made using any suitable techniques and/or any suitable materials for providing an expander with an expanded cross-sectional dimension DE in its relaxed natural state (e.g., the expanded state of assembly 100) and with a reduced cross-sectional dimension DI/DR in its tensioned unnatural state (e.g., the insertion or removal state of assembly 100). Expander 160 may be a valve or any other suitable mechanism that, when expanded within patient 1, may contact a wall of patient 1 for securing expanded assembly 100 at a particular position within patient 1 and/or for preventing certain material from traveling between expander 160 and a wall of patient 1. Expander 160 may be configured to provide a sieve phenomenon such that material (e.g., oropharyngeal secretions, such as saliva) may pass therethrough or thereabout (e.g., from end 161 to end 169 of expander 160 within passageway 15 of patient 1). Expander 160 may be configured such that, in its relaxed natural state (e.g., of FIGS. 1A-1C, 2, 5, and 6-6D), opposing portions of expander 160 defining opposing surfaces 163 for providing cross-sectional dimension DE may be configured to deflect inwardly (e.g., along axis X and axis Z, such as inwardly in an X-Z plane for reducing the cross-sectional size of passageway 165 and dimension DE) when walls of patient 1 may contract or squeeze against expander 160 or otherwise reduce the cross-sectional dimension DO or any other suitable cross-sectional dimension of passageway 15 or target 95. Additionally or alternatively, expander 160 may be configured such that, in its relaxed natural state, opposing portions of expander 160 defining opposing surfaces 163 for providing cross-sectional dimension DE may be configured to rebound outwardly (e.g., along axis X and axis Z, such as outwardly in an X-Z plane for increasing the cross-sectional size of passageway 165 and dimension DE) when walls of patient 1 may open away from expander 160 or otherwise increase the cross-sectional dimension DO or any other suitable cross-sectional dimension of passageway 15 or target 95. Such expansion and contraction of dimension DO of patient 1 may be due to peristalsis of the esophagus or any other suitable portion of patient 1 that may routinely occur during any suitable procedure using assembly 100. By configuring expander 160 to deflect inwardly and rebound outwardly in tandem with expansion and contraction forces of opposing walls of patient 1 about expander 160 may enable expander 160 to safely interact with patient 1 during use. Such inward deflection and outward rebounding of expander 160 while in its natural relaxed state (e.g., of FIGS. 1A-1C, 2, 5, and 6-6D) may or may not alter the spacing between ends 161 and 169 of expander 160 (e.g., dimension ELE). Therefore, expander 160 may be configured to be strong enough in its relaxed state for wall 163 to exert outward pressure against a wall of patient 1 (e.g., to safely maintain a position of expander 160 with respect to a patient wall and/or to safely prevent material from passing therebetween) while also being soft or relaxed enough to be compressed or deflect at least partially inwardly and rebound outwardly for safely enabling contraction and expansion of a wall of patient 1 about expander 160. As mentioned above, such an expander 160 may also be strong enough to pull two tubes (e.g., tubes 120 and 180) closer together (e.g., along the Y-axis) when expander 160 reconfigures itself to its natural state while also being soft enough to deform and rebound (e.g., along the X-axis) when walls of patient 1 contract and expand about expander 160. A balloon is unable to be inflated within a patient with such a degree of sensitivity to perform in this manner, while an expander 160 that may be relaxed in its expanded state may be specifically molded, extruded, and/or otherwise built to perform as desired (e.g., using certain safely performing materials such as silicone, polyurethane, rubber, thermoplastic elastomers, and the like). By enabling two ends of expander 160 to move while expander 160 is positioned within patient 1 may allow for such performance, while a balloon may pop or cause problems to the wall (e.g., necrosis, rupture, etc.) when provided about an intubation assembly tube along a fixed distance of such a tube.

Assembly 100 may enable easy installation and positioning within patient 1 for safe use therein. For example, assembly 100, in its insertion state of FIG. 1, may be positioned within patient 1 such that all of expander 160 may be within target 95 (e.g., such that proximal end 161 of expander 160 is distal of opening 91), and such that, when assembly 100 is thereafter reconfigured from its insertion state to its expanded state, expander 160 may reconfigure itself to its natural expanded state within target 95 (e.g., distal to patient dimension DO). Thereafter, operator O may proximally pull assembly 100 until an expanded portion of expander 160 interacts with a patient wall (e.g., at dimension DO). Doing so with an inflated balloon in its unnatural tensioned expanded state may potentially cause the balloon to pop or otherwise cause trauma when being pulled proximally against a patient wall, and/or inflating a balloon within target 95 may enable balloon to be over-inflated without any reference feedback by a patient wall against the balloon. However, doing so with an expander that is expanded in its relaxed state may enable the expander to be properly and fully expanded within target 95 and then pulled proximally against a patient wall without fear of the expander popping.

If a patient or other operator attempts to remove assembly 100 in its expanded state from patient 1 (e.g., by pulling assembly 100 proximally in the direction of arrow R of FIG. 1A at or near end 101 of assembly 100), assembly 100 may be configured to automatically at least partially reconfigure expander 160 from its natural expanded state to it unnatural tensioned state, thereby reducing dimension DE at least partially towards dimension DI or DR. For example, when assembly 100 in its expanded state is pulled proximally at proximal end 101 in the direction of arrow R (e.g., in the −Y direction), proximal tube 120 may pull proximal end 161 of expander 160 proximally in that same direction. When in its expanded state, expander 160 may at least partially resist such movement due to certain interaction between expander 160 and a wall of patient 1 (e.g., interaction of dimension DE of expander 160 with dimension DO of patient 1), such that proximal pulling of proximal end 161 may increase the distance between proximal end 161 of expander 160 and distal end 169 of expander 160 that may be distal to such interaction of expander 160 with a wall of patient 1 (e.g., for increasing such distance from dimension ELE of the expanded state of assembly 100 at least partially to dimension ELI/ELR of the insertion/removal state of assembly 100), thereby at least partially reconfiguring expander 160 from its natural expanded state to its unnatural insertion/removal state (e.g., at least partially reducing dimension DE to dimension DI/DR) for enabling safe (or at least less traumatic) removal of assembly 100 from patient 1 (e.g., proximal pulling of expander 160 proximally passed dimension DO of patient 1). A balloon would pop or otherwise cause trauma to the tissue wall under such circumstances and would not automatically deflate.

Moreover, while assembly 100 may be positioned within patient 1 for use during any suitable procedure, expander 160 of assembly 100 may be intermittently reconfigured between its natural expanded state and its unnatural state (e.g., through use of deployment mechanism 170). Not only may such intermittent reduction in expander 160 from dimension DE to dimension DI allow material along the outside of assembly 100 to move within passageway 15 along expander 160, but also such intermittent use of deployment mechanism 170 within passageway 115 to do so may allow material within passageway 115 to be moved therealong by mechanism 170 (e.g., distal movement of mechanism 170 within passageway 115 may distally move any material (e.g., food) that may have been lodged or otherwise positioned within passageway 115). A balloon expander mechanism would not enable such action, for example, as repeated intermittent inflation and deflation of a balloon may cause the balloon to lose some of its elasticity. Moreover, by completely removing deployment mechanism 170 from subassembly 110 of assembly 100 once expander 160 is fully expanded may enable a maximum cross-sectional area of passageway 115 of subassembly 110 (e.g., a maximum cross-sectional area of passageway 125 of proximal tube 120) to be used for communicating fluid therethrough to or from target 95 or elsewhere within patient 1. On the other hand, a balloon expander may require an inflation/deflation tube to be constantly available to the balloon during use of the balloon within patient 1, thereby consuming valuable cross-sectional area real estate of the assembly.

Various materials may be used for various elements of an assembly 100, which may vary based on the procedure and/or patient in which assembly 100 is to be used. As just one example, when assembly 100 may be used for a nasogastric intubation procedure, proximal tube 120 and/or distal tube 180 may be made of polyurethane, silicone, polyvinyl chloride, or rubber, deployment tube 140 may be a molded piece and/or extruded piece and/or may be made of nylon and/or may be coupled to distal tube 180 and/or expander 160 via any suitable adhesive (e.g., cyanoacrylate or silicone), expander 160 may be a molded piece and/or extruded piece and/or may be made of silicone, polyurethane, rubber, thermoplastic elastomers, or the like and/or may be coupled to distal tube 180 and/or deployment tube 140 and/or proximal tube 120 via any suitable type of mechanism or bond or adhesive (e.g., cyanoacrylate or silicone glue), and while deploying mechanism 170 may be extruded and/or may be made of nylon, polytetrafluoroethylene (e.g., as a rod or tube) and/or may include a lubricious coating for easy passage through passageway 115. One or more of expander 160, tube 120, tube 140, tube 180, and the like may be provided with an alkaline coating on one or both of its interior and exterior walls, such that when material (e.g., food or acidic stomach contents) travels through such components, the acidity of the material may get neutralized. Additionally or alternatively, one or more of expander 160, tube 120, tube 140, tube 180, and the like may be at least partially X-ray visible such that an operator may ensure that it is properly placed within patient 1 for a particular procedure.

Assembly 100 may have any suitable dimensions, such that assemblies of different dimensions may be used for different procedures within patient 1 and/or for the same procedure within different patients of different sizes. As just one example, when assembly 100 may be used for a nasogastric intubation procedure on an adult male, length ELE of expander 160 in its relaxed and expanded state may be about 51 millimeters (e.g., between ends 161 and 169) and/or length ELE* of expander 160 in its relaxed and expanded state may be about 30 millimeters (e.g., between expandable ends 161* and 169* of FIG. 6B), while dimension DE of expander 160 may be about 22 millimeters, while dimension DI of expander 160 may be about 5.7-6.0 millimeters, while thickness dimension DT of expander 160 of FIG. 6C may be about 0.2 millimeters, while thickness dimension DTT of expander 160 of FIG. 6B may be about 5.5 millimeters, while overall length LE of assembly 100 may be about 1240 millimeters, while distal tube 180 may extend 180-260 millimeters beyond distal end 169 of expander 160, while dimension DP and/or DD may be about 5.5 millimeters, while a thickness of deployment tube 140 may be about 0.5 millimeters and/or may have a cross-sectional diameter of about 4.2 millimeters and/or a length of about 77 millimeters, and while deploying mechanism 170 may have a cross-sectional diameter of about 1.8 millimeters and/or may have a length of about 955.5 millimeters.

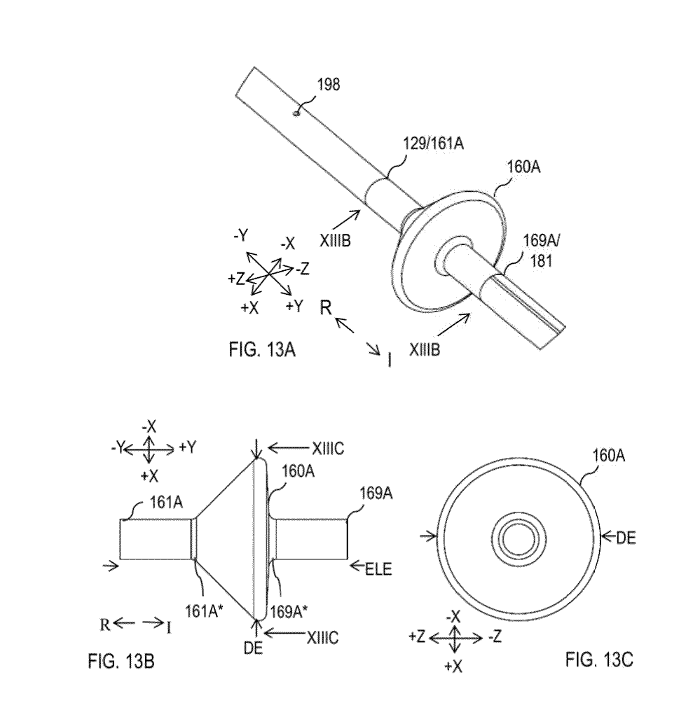

While expander 160 of FIGS. 1-12 may be shown as provided with a double conical shape in its natural relaxed expanded state (e.g., with a first conical shape expanding in dimension distally away from proximal end 161/161* to a middle section of expanded dimension DE and with a second conical shape expanding in dimension proximally away from distal end 169/169* towards such a middle section of expanded dimension DE (see, e.g., FIG. 6B)), an expander of assembly 100 may be configured to be of any other suitable shape in its natural relaxed expanded state. For example, as shown in FIGS. 13A-13C, assembly 100 may instead be provided with an expander 160A that may be provided with a single conical shape in its natural relaxed expanded state (e.g., with a single conical shape expanding in dimension distally away from proximal end 161A/161A* to a middle section of expanded dimension DE that may abut distal end 169* (see, e.g., FIG. 13B)). Like expander 160, expander 160A may also provide a sloped distally expanding interface at its proximal end to a dimension DE, where such a sloped interface may safely interact with a dimension DO of patient 1. Such a sloped interface may be gentle and not abrupt so as not to erode or puncture a patient wall during interaction therewith. Other examples may include a conical shape proximally with a square bottom distally, and a double conical shape with both conical shapes expanding proximally, which may allow any materials coming up from the stomach to be better blocked.

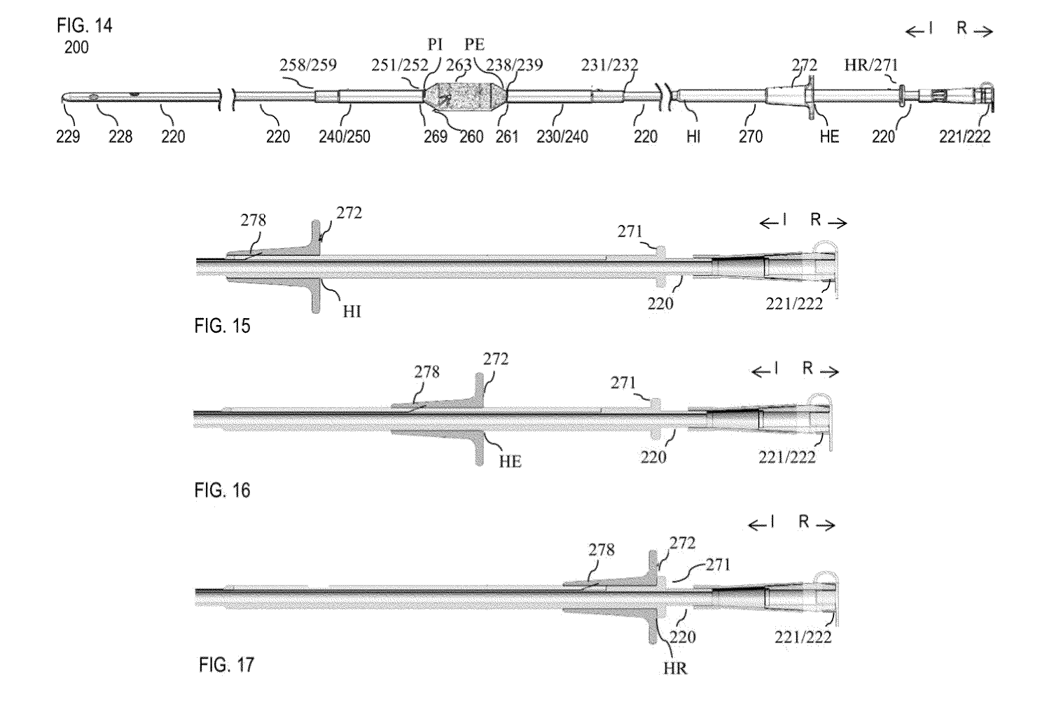

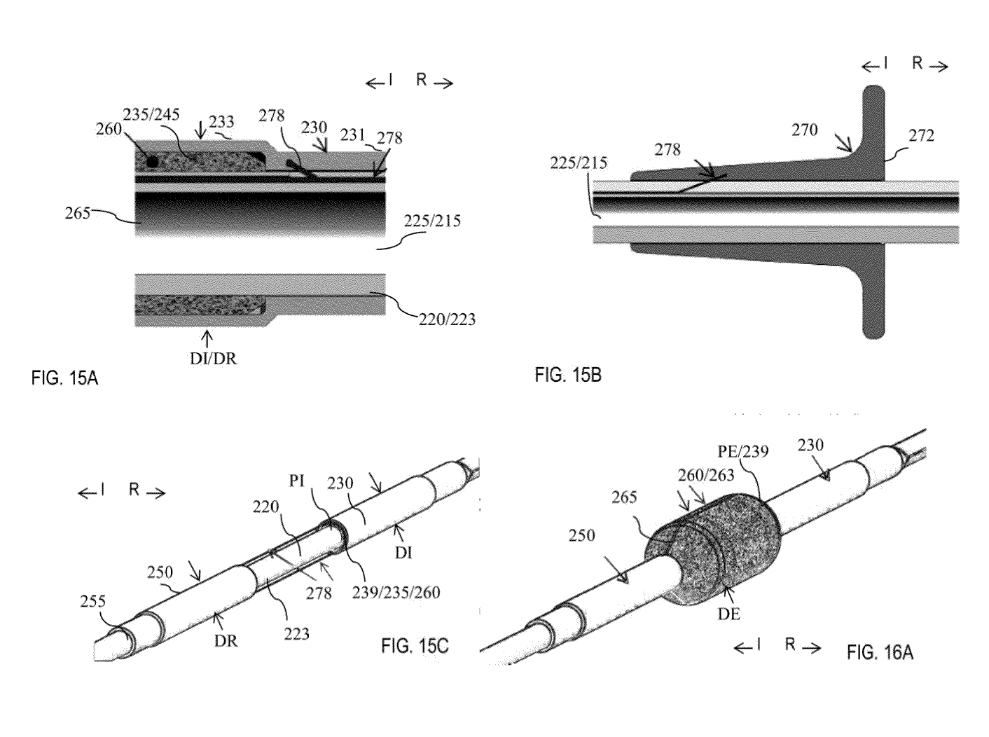

FIGS. 14-17 show an illustrative assembly 200 in different configurations or stages of use for any suitable procedure with respect to patient 1 of FIGS. 1-1D, similarly to assembly 100 of FIGS. 1-13C. As shown in FIGS. 14-17, in some embodiments, assembly 200 may include a first or inner tube subassembly 220, a second or outer deployment tube subassembly 240, an expander 260, and a deployment mechanism 270. For example, inner tube subassembly 220 may extend between a proximal or first end 221 (e.g., assembly end 101 of FIGS. 1-1D) and a distal or second end 229 (e.g., assembly end 109 of FIGS. 1-1D) Inner tube 220 may include at least one tube wall 223 that may define at least one internal passageway 225 (e.g., at least a portion of passageway 115 of FIGS. 1-1D) extending along at least a portion of assembly 200. Wall 223 may also include at least one proximal or first tube opening 222 (e.g., opening 102 of FIGS. 1-1D) that may provide access to passageway 225 at or near end 221 of assembly 200 and at least one distal or second tube opening 228 that may provide access to passageway 225 at or near end 229 of assembly 200. As shown, expander 260 may include a wall defining an external surface 263 and an expander passageway 265 that may extend between a first or proximal expander end 261 and a second or distal expander end 269. A wall defining external surface 263 may also include at least one proximal or first expander opening 262 that may provide access to passageway 265 at or near end 261 of expander 260 and at least one distal or second expander opening 268 that may provide access to passageway 265 at or near end 269 of expander 260. A portion of tube 220 may extend through passageway 265 of expander 260 (e.g., such that an interior wall of expander 260 defining passageway 265 may be coupled to a portion of exterior wall 223 of tube 220 (see, e.g., FIG. 15A)). Outer deployment tube subassembly 240 may include a first or proximal outer tube 230 and a second or distal outer tube 250. Alternatively, outer tubes 230 and 250 may be provided as a single tube (e.g., a single tube with one or more openings provided along its side for enabling expander 260 to expand therethrough (e.g., a single tube embodiment of outer deployment tube subassembly 240 may have a similar shape to the combined shape of tubes 230 and 250 but where wires 278 extending between 230 and 250 are instead portions of the single tube)). Proximal outer tube 230 may include at least one tube wall 233 that may define at least one internal passageway 235 extending along at least a portion of assembly 200 about at least a portion of tube 220. Wall 233 may also include at least one proximal or first tube opening 232 that may provide access to passageway 235 at or near end 231 of proximal outer deployment tube 230 and at least one distal or second tube opening 238 that may provide access to passageway 235 at or near end 239 of proximal outer deployment tube 230. Distal outer tube 250 may include at least one tube wall 253 that may define at least one internal passageway 255 extending along at least a portion of assembly 200 about at least a portion of tube 220. Wall 253 may also include at least one proximal or first tube opening 252 that may provide access to passageway 255 at or near end 251 of distal outer deployment tube 250 and at least one distal or second tube opening 258 that may provide access to passageway 255 at or near end 259 of distal outer deployment tube 250. Deployment mechanism 270 may include a handle 272 at or near a proximal end of assembly 200 (e.g., for use by operator O) and any suitable adjustment mechanism (e.g., wire) 278 that may extend from handle 272 to outer deployment tube subassembly 240 (e.g., for moving outer deployment tube subassembly 240 about and along inner tube subassembly 220, which may re-configure expander 260 between a relaxed natural expanded state and an unnatural tensioned restricted state).

Expander 260 may be coupled about a portion of tube subassembly 220 (e.g., between ends 221 and 229) or may fluidly couple two distinct tubes of subassembly 220 (e.g., similarly to expander 160, which may fluidly couple tubes 120 and 180 of FIG. 6). As described below in more detail, expander 260 may be operative to be reconfigured between a first natural or relaxed state and a second unnatural or tensioned state. For example, as shown in FIGS. 1A, 14, and 16A, expander 260 may be in a natural or relaxed state when at least a portion of expander 260 is not retained within a portion of outer deployment tube subassembly 240 such that at least a portion of expander 260 may have a maximum cross-sectional dimension (e.g., diameter) DE, which may be at least equal to or greater than dimension DO of patient 1 (e.g., as described above, such that at least a portion of wall 263 of expander 260 may contact or otherwise interact with at least a portion of a wall of patient 1 for safely securing expanded assembly 200 at a particular position within patient 1 and/or for safely preventing certain material from traveling between wall 263 of expander 260 and at least a portion of a wall of target 95 and/or passageway 15 of patient 1), such that such a natural or relaxed state of expander 260 may be used for an expanded state of assembly 200 within patient 1. However, as shown in FIGS. 1, 1D, 15A, and 15C, for example, expander 260 may be in an unnatural or tensioned state when at least a portion of expander 160 may be retained within a portion of outer deployment tube subassembly 240, whereby no portion of expander 260 in combination with outer deployment tube subassembly 240 may have a cross-sectional dimension (e.g., diameter) greater than dimension DI and/or DR, which may be less than dimension DO of patient 1 (e.g., as described above), such that such an unnatural or tensioned state of expander 260 may be used for an insertion state into patient 1 and/or a removal state of assembly 200 from within patient 1. Assembly 200 may be provided with any suitable components or features for reconfiguring expander 260 between its natural and un-natural states (e.g., between the expanded and insertion/removal states of assembly 200).

In some embodiments, outer deployment tube subassembly 240 may be positioned about tube assembly 220 such that deployment mechanism 270 may be configured to move outer deployment tube subassembly 240 along subassembly 220 for adjusting the amount of expander 260 (e.g., length of expander 260 between ends 261 and 269) retained between outer deployment tube subassembly 240 and inner subassembly 220, thereby reconfiguring expander 260 between its natural and un-natural states (e.g., thereby reconfiguring assembly 200 between its expanded state and insertion/removal state). For example, as shown in FIGS. 1 and 15-15C, at least a portion or all of expander 260 may be positioned between an exterior of wall 223 of inner tube assembly 220 and an interior of a wall defining exterior 233 of proximal outer deployment tube assembly 230, such that no portion of expander 260 in combination with outer deployment tube subassembly 240 may have a cross-sectional dimension (e.g., diameter) greater than dimension DI and/or DR, which may be less than dimension DO of patient 1 (e.g., as described above). A portion of proximal outer deployment tube assembly 230 (e.g., at or near proximal end 231) may be coupled to deployment mechanism 270 (e.g., to a portion of adjustment mechanism 278). As shown, adjustment mechanism 278 may be a wire or any suitable feature of deployment mechanism 270 that may be coupled to both handle 272 and to deployment subassembly 240 (e.g., proximal outer subassembly 230 and/or distal outer subassembly 250).

When in an initial or first position, handle 272 of deployment mechanism 270 may be at a first or distal position HI along assembly 200, such that adjustment mechanism 278 may enable deployment subassembly 240 to cover or otherwise retain expander 260 in its insertion state (e.g., an unnatural tensioned state in which expander 260 is not expanded for providing dimension DE). For example, as shown, in such an insertion state of assembly 200, at least a portion if not all of expander 260 may be retained by subassembly 230 between subassembly 230 and subassembly 220 (e.g., within passageway 235 between ends 231 and 239) for providing dimension DI.

Then, as shown in FIGS. 1A, 14, 16, and 16A, handle 272 of deployment mechanism 270 may be pulled (e.g., proximally in the direction of arrow R) from position HI to position HE, such that adjustment mechanism 278 may pull at least a portion of subassembly 240 (e.g., subassembly 230 and/or subassembly 250) proximally (e.g., in the direction of arrow R) such that at least a portion of expander 260 may be enabled to reconfigure to its expanded state, thereby providing dimension DE. As shown, this may pull end 239 of subassembly 230 (e.g., along subassembly 220) from point PI to point PE, which may be equal to the length of expander 260 and/or the length from point HI to point HE. For example, as shown, in such an expanded state of assembly 200, at least a portion if not all of expander 260 may be positioned between subassembly 230 and subassembly 250 yet not retained by either of subassemblies 230 or 250 (e.g., between ends 239 and 251), such that expander may provide dimension DE.

Then, as shown in FIGS. 1D and 17, handle 272 of deployment mechanism 270 may be pulled (e.g., proximally in the direction of arrow R) from position HE to position HR, such that handle 272 and adjustment mechanism 278 may pull at least a portion of subassembly 240 (e.g., subassembly 230 and/or subassembly 250) proximally (e.g., in the direction of arrow R) to cover or otherwise retain expander 260 in its removal state (e.g., an unnatural tensioned state in which expander 260 is not expanded for providing dimension DE), such that at least a portion of expander 260 may be enabled to reconfigure to its restricted unexpanded state, thereby providing dimension DR. For example, similarly to as shown in FIGS. 15A and 15C when expander 260 may be disposed between subassemblies 220 and 230, in such a removal state of assembly 200, at least a portion if not all of expander 260 may be retained by subassembly 250 between subassembly 250 and subassembly 220 (e.g., within passageway 255 between ends 251 and 259) for providing dimension DR. An element 271 may be positioned along subassembly 220 at point HR to indicate when handle 272 is at point HR, such that an operator may know when expander 260 has been reconfigured to its restricted state within patient 1, such that assembly 200 may be safely removed from patient 1.

As shown in FIG. 15C, for example, a portion of adjustment mechanism 278 may extend between ends 239 and 251, such as one or two or more wires or other suitable elements, such that movement of subassembly 230 along assembly 200 (e.g., in the direction of arrow R) may pull or similarly move subassembly 250. As shown in FIG. 16A, for example, one or more associated features (e.g., slits) may be provided through a portion of exterior 263 of expander 260, which may enable such portion(s) of adjustment mechanism 278 to pass through at least a portion of expander 260 (e.g., when at least a portion of expander 260 is positioned between ends 239 and 251 in the expanded state of expander 260). This may enable expander 260 to expand while still enabling adjustment mechanism 278 to couple subassemblies 230 and 250. Additionally or alternatively, as shown in FIG. 15A, for example, at least a portion of adjustment mechanism 278 may pass along assembly 200 within a portion of passageway 215, 225, 235, and/or 245.

Various materials may be used for expander 260 of assembly 200, such as foam, sponger, or any other suitable material that may be restricted into an unnatural tensioned state at least partially within a passageway of deployment subassembly 240 between subassembly 240 and subassembly 220 when subassembly 240 is moved along and about subassembly 220 and expander 260 that may be coupled to a specific portion of subassembly 220. The length of expander 260 may be any suitable length, such as 20 millimeters. Unlike a balloon expander, expander 260 may be at least partially made of a foam or any other suitable material that may not cause pressure on the patient wall (e.g., esophageal wall) and that may be able to partially revert to its unnatural state if the wall contracts, thereby minimizing the risk of wall necrosis and wall rupture. Such an expander 260 may also be configured to soak any saliva or other fluids that may contact expander 260 and may be eventually released from expander 260 (e.g., with forward peristalsis). The total diameter (e.g., dimension DE) of expander 260 may be about 20 millimeters in its expanded state. When expander 260 is reconfigured from its expanded state to its removal state, some or all of the fluids (e.g., saliva) that have been soaked into or otherwise retained by expander 260 may be expelled therefrom (e.g., such that the fluids may pass down the esophagus and into the stomach).

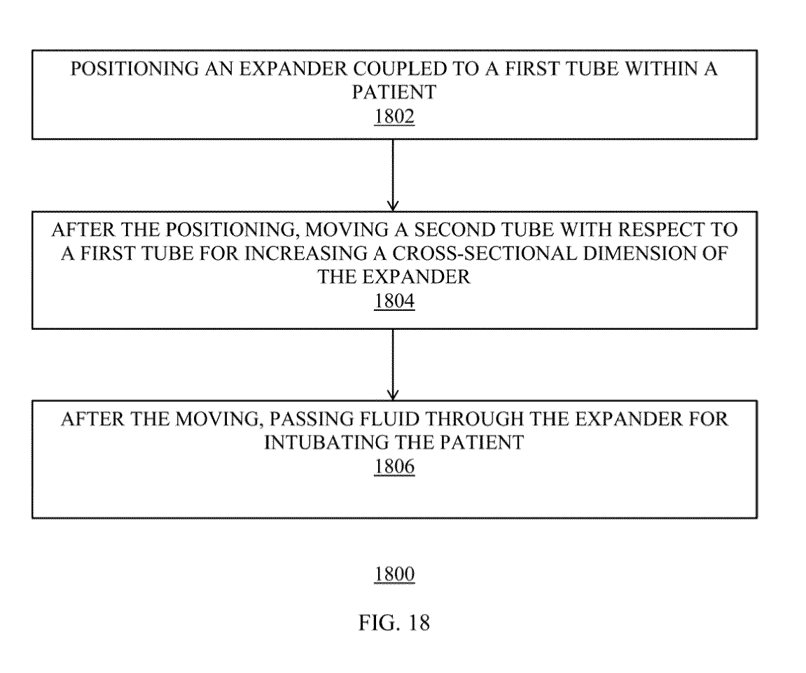

FIG. 18 is a flowchart of an illustrative process 1800 for intubating a patient with an assembly, where the assembly may include a first tube, a second tube, and an expander coupled to the first tube. At step 1802 of process 1800, the expander may be positioned within the patient. For example, as described above and shown in FIG. 1, an assembly 100, which may include expander 160 coupled to proximal tube 120 of FIGS. 2-6D or an expander 260 coupled to tube 220 of FIGS. 14-17, may be positioned within patient 1. Then, at step 1804, after the positioning of step 1802, process 1800 may include moving the second tube with respect to the first tube for increasing a cross-sectional dimension of the expander. For example, as described above and shown in FIGS. 1A and 6-6D, deployment tube 140 may be moved with respect to proximal tube 120 (e.g., by at least distance D of FIG. 4) for increasing a cross-sectional dimension of expander 160 from dimension DI to dimension DE. As another example, as described above and shown in FIGS. 1A and 14-17, tube 230 may be moved with respect to tube 220 (e.g., by at least the distance between points PI and PE of FIGS. 14, 15C, and 16A) for increasing a cross-sectional dimension of expander 260 from dimension DI to dimension DE. Then, at step 1806, after the moving of step 1804, process 1800 may include passing fluid through the expander for treating the patient. For example, as described above and shown in one or more of FIGS. 1A-1C, after expander 160/260 has been expanded, fluid may be passed through passageway 115 (e.g., through expander passageway 165/265) for treating patient 1 (e.g., at target 95).

It is understood that the steps shown in process 1800 of FIG. 18 are merely illustrative and that existing steps may be modified or omitted, additional steps may be added, and the order of certain steps may be altered.

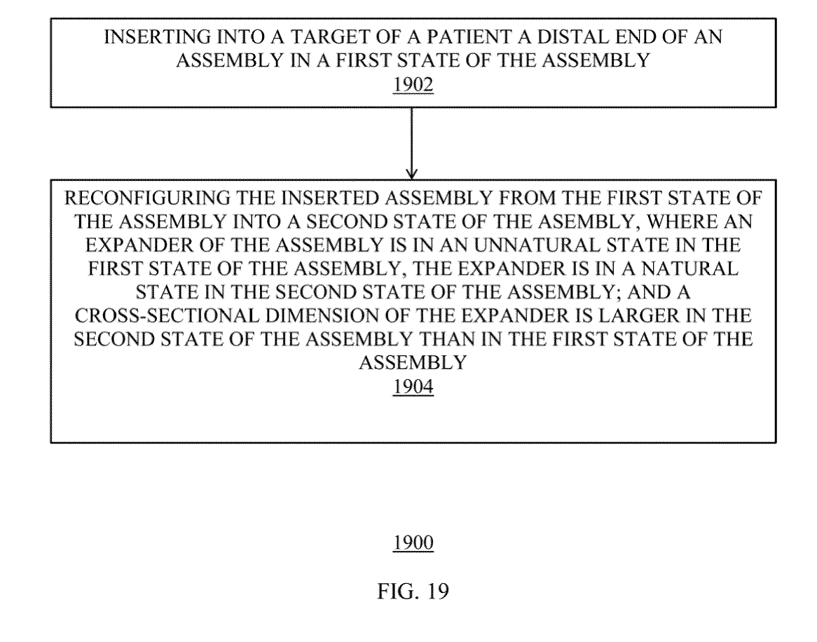

FIG. 19 is a flowchart of an illustrative process 1900 for intubating a target of a patient via a passageway of the patient with an assembly, where a length of the assembly may extend between a proximal end and a distal end and may include an expander. At step 1902 of process 1900, the distal end of the assembly in a first state of the assembly may be inserted into the target, such that the expander is at least partially within one of the target and the passageway. For example, as described above and shown in FIG. 1, distal end 109 of assembly 100, which may include expander 160 of FIGS. 2-6D or expander 260 of FIGS. 14-17, may be inserted into target 95 of patient 1 while assembly 100 is in its insertion state. Then, at step 1904, after the inserting of step 1902, process 1900 may include reconfiguring the inserted assembly from the first state of the assembly into a second state of the assembly, where the expander is in an unnatural state in the first state of the assembly, the expander is in a natural state in the second state of the assembly, and a cross-sectional dimension of the expander is larger in the second state of the assembly than in the first state of the assembly. For example, as described above and shown in one or more of FIGS. 1A-1C, assembly 100 may be reconfigured from its insertion state into an expanded state, where a cross-sectional dimension DE of expander 160 in the expanded state of assembly 100 is larger than cross-sectional dimension DI of expander 160 in the insertion state of assembly 100 of FIG. 1. As described above with respect to expander 160 of assembly 100 of FIGS. 2-6D, expander 160 may be in a natural relaxed expansion state when assembly 100 is in its expansion state and in an unnatural tensioned state when assembly 100 is in its insertion state. Similarly, as described above with respect to expander 260 of assembly 200 of FIGS. 14-17, expander 260 may be in a natural relaxed expansion state when assembly 200 is in its expansion state (e.g., when expander 260 may be able to expand without being covered by subassembly 240) and in an unnatural tensioned state when assembly 200 is in its insertion state (e.g., when expander 260 may be deformed within a passageway of subassembly 240).

It is understood that the steps shown in process 1900 of FIG. 19 are merely illustrative and that existing steps may be modified or omitted, additional steps may be added, and the order of certain steps may be altered.

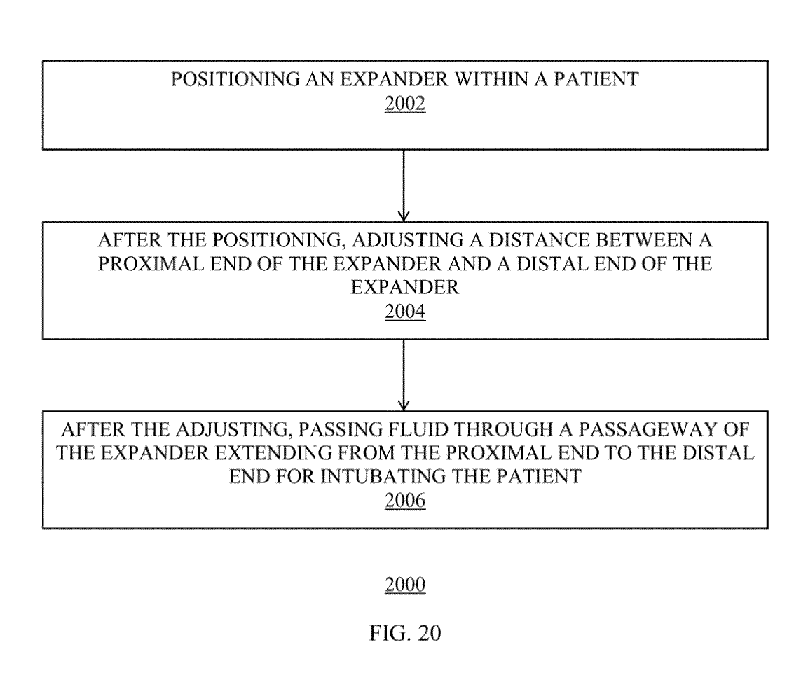

FIG. 20 is a flowchart of an illustrative process 2000 for intubating a patient with an assembly that may include an expander, where the expander may include an expander passageway extending from a proximal expander end to a distal expander end. At step 2002 of process 2000, the expander may be positioned within the patient. For example, as described above and shown in FIG. 1, expander 160 may be positioned within patient 1. Then, at step 2004, after the positioning of step 2002, process 2000 may include adjusting a distance between the proximal expander end and the distal expander end. For example, as described above and shown in one or more of FIGS. 1A-1C and 2-6D, a distance between ends 161 and 169 of expander 160 may be adjusted (e.g., from distance ELI of FIG. 4 to distance ELE of FIG. 6). Then, at step 2006, after the adjusting of step 2004, process 2000 may include passing fluid through the expander for treating the patient. For example, as described above and shown in one or more of FIGS. 1A-1C, after expander 160 has been expanded (e.g., through adjustment of the distance between ends 161 and 169 of expander 160 from distance ELI to distance ELE), fluid may be passed through passageway 115 (e.g., through expander passageway 165) for treating patient 1.

It is understood that the steps shown in process 2000 of FIG. 20 are merely illustrative and that existing steps may be modified or omitted, additional steps may be added, and the order of certain steps may be altered.

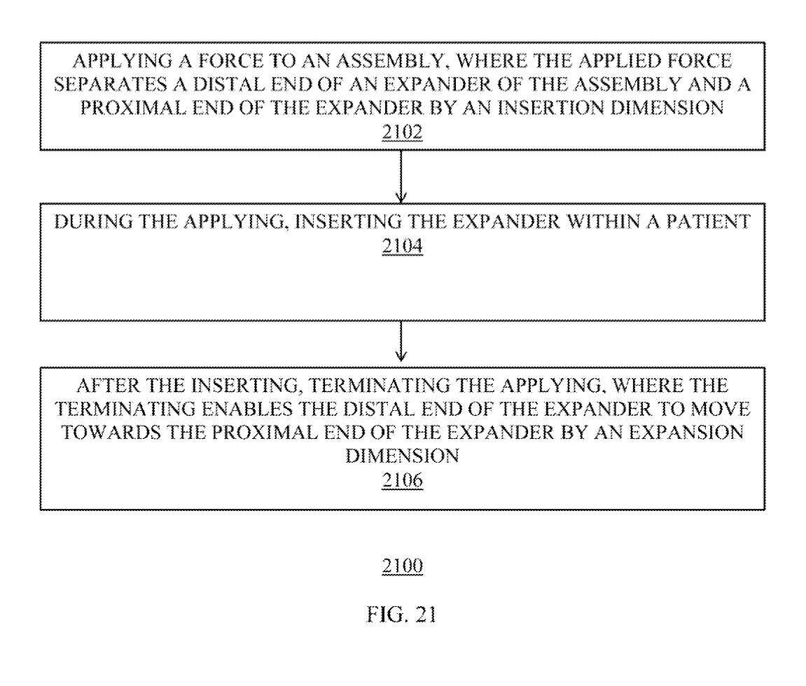

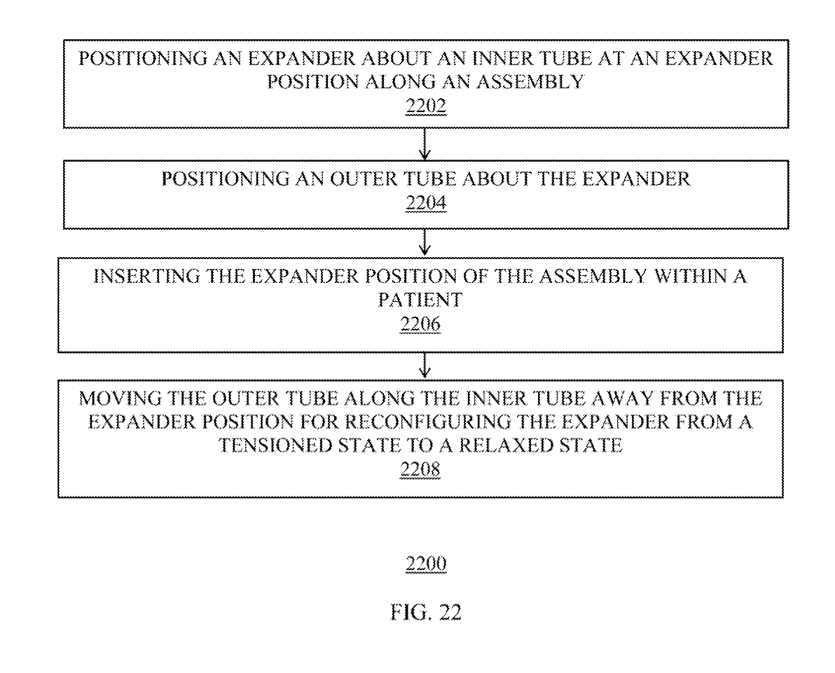

FIG. 21 is a flowchart of an illustrative process 2100 for intubating a patient with an assembly that may include an expander, where the expander may include an expander passageway extending from a proximal expander end to a distal expander end. At step 2102 of process 2100, a force may be applied to the assembly, where the applied force may separate the distal expander end and the proximal expander end by an insertion dimension. For example, as described above and shown in one or more of FIGS. 1, 4, and 4A, a force may be applied on assembly 100 (e.g., by mechanism 170) that may separate ends 161 and 169 of expander 160 by a distance ELI. Then, at step 2104, during the applying of step 2102, process 2100 may include inserting the expander within the patient. For example, as described above and as shown in FIG. 1, when assembly 100 is in its insertion state of FIGS. 4 and 4A, assembly 100 may be inserted into patient 1. Then, at step 2106, after the inserting of step 2104, process 2100 may include terminating the application of force of step 2102, where the termination of the applied force may enable the distal expander end to move towards the proximal expander end by an expansion dimension. For example, as described above and shown in one or more of FIGS. 1A-1C and 2-6D, when a force on expander 160 may be terminated (e.g., by at least partially removing mechanism 170 proximally from assembly 100), ends 161 and 169 of expander 160 may move towards each other by a distance (e.g., the difference between ELI and ELE, or distance D of FIG. 4).

It is understood that the steps shown in process 2100 of FIG. 21 are merely illustrative and that existing steps may be modified or omitted, additional steps may be added, and the order of certain steps may be altered.

FIG. 22 is a flowchart of an illustrative process 2200 for intubating a patient with an assembly that may include an inner tube, an outer tube, and an expander. At step 2202 of process 2200, the expander may be positioned about the inner tube at an expander position along the assembly. For example, as described above with respect to FIGS. 14-17, expander 260 may be positioned about tube 220 (e.g., between positions PI and PE) of assembly 200. Then, at step 2204, process 2200 may include positioning the outer tube about the expander. For example, as described above with respect to FIGS. 14-17, subassembly 250 (e.g., outer tube 230) may be positioned about expander 260 (e.g., as shown in FIGS. 15A and 15C). Then, at step 2206, the expander position of the assembly may be inserted within the patient. For example, as described above and as shown in FIG. 1, when assembly 200 is in its insertion state of FIGS. 15-15C, the expander of assembly 200 may be inserted into patient 1. Then, at step 2208, after the inserting of step 2206, process 2200 may include moving the outer tube along the inner tube away from the expander position for reconfiguring the expander from a tensioned state to a relaxed state. For example, as described above with respect to FIGS. 1A and 14-17, when expander 260 is positioned within patient 1, subassembly 250 (e.g., outer tube 230) may be moved along inner tube 220 proximally away from expander 260 (e.g., in the direction of arrow R) for reconfiguring expander 260 from a tensioned state (e.g., a deformed state within passageway 235) to a relaxed state (e.g., free from passageway 235).

It is understood that the steps shown in process 2200 of FIG. 22 are merely illustrative and that existing steps may be modified or omitted, additional steps may be added, and the order of certain steps may be altered.

While there have been described expandable assemblies and methods for using and making the same, it is to be understood that many changes may be made therein without departing from the spirit and scope of the subject matter described herein in any way. Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. It is also to be understood that various directional and orientational terms such as “proximal” and “distal,” “up” and “down,” “front” and “back,” “top” and “bottom” and “side,” “length” and “width” and “thickness” and “diameter” and “cross-section” and “longitudinal,” “X-” and “Y-” and “Z-,” and the like that may be used herein only for convenience, and that no fixed or absolute directional or orientational limitations are intended by the use of these words. For example, the assemblies and patients can have any desired orientations. If reoriented, different directional or orientational terms may need to be used in their description, but that will not alter their fundamental nature as within the scope and spirit of the subject matter described herein in any way.

Therefore, those skilled in the art will appreciate that the invention can be practiced by other than the described embodiments, which are presented for purposes of illustration rather than of limitation.

1. An intubation assembly comprising:
a first tube comprising a first tube passageway extending from a proximal first tube end to a distal first tube end along a first tube portion of the length of the assembly; and
an expander comprising an expander passageway extending from a proximal expander end to a distal expander end along an expander portion of the length of the assembly, wherein:
the proximal expander end is coupled to the distal first tube end; and
movement of the distal expander end with respect to the proximal expander end along the length of the assembly adjusts a cross-sectional dimension of the assembly.

2. The intubation assembly of claim 1, wherein:
when the expander is in a natural state, the proximal expander end is separated from the distal expander end by a first distance along the length of the assembly;
when the expander is in the natural state, the maximum cross-sectional dimension of the expander is a first width;
when the expander is in an unnatural state, the proximal expander end is separated from the distal expander end by a second distance along the length of the assembly;
when the expander is in the unnatural state, the maximum cross-sectional dimension of the expander is a second width;
the first distance is shorter than the second distance; and
the first width is greater than the second width.

3. The intubation assembly of claim 1, further comprising a deployment mechanism that passes through the first tube passageway and through at least a portion of the expander passageway for moving the distal expander end away from the proximal expander end.

4. The intubation assembly of claim 1, further comprising a deployment tube comprising a deployment tube passageway extending from a proximal deployment tube end to a distal deployment tube end, wherein:
the distal deployment tube end is coupled to the distal expander end;
the proximal deployment tube end is positioned within the first tube passageway; and
movement of the proximal deployment tube end with respect to the proximal first tube end adjusts a cross-sectional dimension of the expander.

5. The intubation assembly of claim 4, further comprising a deployment mechanism that passes through at least a portion of the first tube passageway for moving the proximal deployment tube end away from the proximal first tube end.

6. The intubation assembly of claim 1, further comprising a second tube comprising a second tube passageway extending from a proximal second tube end to a distal second tube end along a second tube portion of the length of the assembly, wherein:
the proximal second tube end is coupled to the distal expander end;
the first tube comprises a first tube opening proximate the proximal first tube end;
the second tube comprises a second tube opening proximate the distal second tube end; and
the intubation assembly is operative to communicate material between the first tube opening and the second tube opening through at least a portion of the first tube passageway, through the expander passageway, and through at least a portion of the second tube passageway.

7. The intubation assembly of claim 1, further comprising:
a second tube comprising a second tube passageway extending from a proximal second tube end to a distal second tube end along a second tube portion of the length of the assembly; and
a deployment component extending from a proximal deployment component end to a distal deployment component end, wherein:
a distal portion of the deployment component is fixed with respect to the distal expander end and the proximal second tube end; and
movement of a proximal portion of the deployment component with respect to the proximal expander end adjusts a cross-sectional dimension of the expander.

8. The intubation assembly of claim 7, wherein the intubation assembly is operative to communicate material between the first tube passageway and the second tube passageway via the expander passageway.

9. The intubation assembly of claim 7, wherein:
the deployment component comprises a deployment tube passageway extending between the proximal deployment component end and the distal deployment component end;
at least a portion of the deployment tube passageway extends through the expander passageway; and
the intubation assembly is operative to communicate material between the first tube passageway and the second tube passageway via the deployment tube passageway.

10. An intubation assembly comprising:
a first tube comprising a first tube passageway extending from a proximal first tube end to a distal first tube end along a first tube portion of the length of the assembly;
a second tube comprising a second tube passageway extending from a proximal second tube end to a distal second tube end along a second tube portion of the length of the assembly; and
an expander comprising an expander passageway extending from a proximal expander end to a distal expander end along an expander portion of the length of the assembly, wherein:
the expander is coupled to the first tube; and
movement of the second tube with respect to the first tube along the length of the assembly adjusts a cross-sectional dimension of the expander.

11. The intubation assembly of claim 10, wherein the movement of the second tube with respect to the first tube along the length of the assembly enables reconfiguration of the expander between a relaxed expander state and a tensioned expander state.

12. The intubation assembly of claim 10, wherein:
the proximal expander end is coupled to the distal first tube end;
the distal expander end is coupled to the second tube; and
the second tube is operative to move within the expander passageway for adjusting the distance between the proximal expander end and the distal expander end.

13. The intubation assembly of claim 10, wherein:
the first tube passageway extends through the expander passageway along the expander portion of the length of the assembly;
the second tube is operative to move with respect to the first tube along the length of the assembly to vary the amount of the expander portion of the length of the assembly that is positioned within the second tube passageway.

14. The assembly of claim 10, wherein rotation of the second tube with respect to the first tube about a longitudinal axis of the length of the assembly adjusts the cross-sectional dimension of the expander.

15. A method of intubating a patient with an assembly comprising an expander, a first tube, and a second tube, wherein the expander is coupled to the first tube, the method comprising:
positioning the expander within the patient;
after the positioning, moving the second tube with respect to the first tube for increasing a cross-sectional dimension of the expander; and
after the moving, passing fluid through the expander for treating the patient.

16. The method of claim 15, wherein the moving enables reconfiguration of the expander from an unnatural expander state to a natural expander state.

17. The method of claim 15, wherein:
the expander comprises an expander passageway extending from a proximal expander end to a distal expander end along a longitudinal axis of the expander;
the proximal expander end is coupled to the first tube;
the distal expander end is coupled to the second tube; and
the moving comprises rotating the second tube with respect to the first tube about the longitudinal axis of the expander.

18. The method of claim 15, wherein the moving comprises moving the second tube from a first position where a portion of the expander is deformed between the first tube and the second tube to a second position where the portion of the expander is not between the first tube and the second tube.

19. The method of claim 15, wherein:
the first tube comprises a first tube passageway extending from a proximal first tube end to a distal first tube end along a first tube portion of the length of the assembly;
the second tube comprises a second tube passageway extending from a proximal second tube end to a distal second tube end along a second tube portion of the length of the assembly;
the expander comprises an expander passageway extending from a proximal expander end to a distal expander end along an expander portion of the length of the assembly;
the first tube passageway extends through the expander passageway along the expander portion of the length of the assembly; and
the moving comprises moving the second tube with respect to the first tube along the length of the assembly to vary the amount of the expander portion of the length of the assembly that is positioned within the second tube passageway.

20. The method of claim 15, wherein:
the expander comprises an expander passageway extending from a proximal expander end to a distal expander end;
the proximal expander end is coupled to the first tube;
the distal expander end is coupled to the second tube; and
the moving comprises moving the second tube within the expander passageway for adjusting the distance between the proximal expander end and the distal expander end.

21.-27. (canceled)
